(19) United States
(12) Patent Application Publication
(10) Pub. No.: US 2002/0116135 A1
(43) Pub. Date: Aug. 22, 2002

(54) Methods, systems, and articles of manufacture for evaluating biological data

(76) Inventors: Hugh J. Pasika, San Francisco, CA (US); Yuandan Lou, Cupertino, CA (US); David P. Holden, San Francisco, CA (US); Heinz Breu, Palo Alto, CA (US)

Correspondence Address: Finnegan, Henderson, Farabow,, Garrett & Dunner, L.L.P., 1300 I Street, N.W., Washington, DC, 20005-3315, US

(21) Appl. No.: 09/911,903

(22) Filed: Jul. 23, 2001

Related U.S. Application Data
(60) Provisional application No. 60/219,697, filed on Jul. 21, 2000.
(60) Provisional application No. 60/227,556, filed on Aug. 23, 2000.
(60) Provisional application No. 60/290,129, filed on May 10, 2001.

(51) Int. Cl. G06F 19/00; G01N 33/48; G01N 33/50

(52) U.S. Cl. 702/21; 702/19

(57) ABSTRACT

Methods, systems, and articles of manufacture consistent with the present invention are provided for making allele calls. In certain embodiments, allele calling is accomplished by providing a committee machine that receives calls from several allele calling algorithms. By receiving calls from multiple allele calling algorithms, the committee machine makes calls containing a high level of confidence over a variety of conditions. Certain embodiments provide methods employing at least two algorithms and at least two quality values for allele calling. Unique individual algorithms for allele calling are also provided.

This application claims the benefit of U.S. Provisional Application Serial No. 60/219,697, filed on Jul. 21, 2000, U.S. Provisional Application Serial No. 60/227,556, filed on Aug. 23, 2000, U.S. application Ser. No. 09/724,910, filed Nov. 28, 2000, and U.S. Provisional Application Serial No. 60/290,129, filed on May 10, 2001. This application incorporates by reference all of the disclosure of U.S. Ser. Nos. 60/219,697, 60/227,556, 09/724,910, and 60/290,129 for any purpose.

### FIELD OF THE INVENTION

This invention relates to data methods and systems for assigning values to nucleic information. In certain embodiments, the methods and systems are used for assigning values to alleles.

### BACKGROUND OF THE INVENTION

There are many techniques for analyzing nucleic acid information. For example, certain techniques involve studying genetic polymorphisms. A polymorphism involves difference in a given portion of a nucleic acid sequence in different individuals within a population. Such polymorphisms may occur in regions in which nucleic acids do not encode proteins. In such regions, often there are large numbers of repeats of a given short sequence. For example, there may be regions of multiple repeats of a given dinucleotide (such as GC or CA), trinucleotides, or larger repeat units. The larger repeat regions (larger number of nucleotide bases within a repeated motif) have often been referred to as “minisatellites.” The smaller repeat regions (1, 2, 3, 4, 5, or 6 nucleotides within a repeated motif) have often been referred to as “microsatellites” or “short tandem repeats (STR's).” Through evolution, individuals often vary in the number of repeats at a given locus.

Such repeat regions can serve as genetic markers since individuals can vary in the number of repeats at a given locus (location) or at many loci (locations). Each different form at a given locus is known as an allele. These differences at a given position can serve as genetic markers that are useful for many purposes including positively identifying an individual from genetic material based on the unique genetic pattern of such an individual.

Also, variations between individuals may signify predisposition to a disease or other genetic conditions. Linkage studies also involve determination of alleles.

Thus, much effort has been focused on positively identifying particular alleles at given genetic loci. For example, methods of determining the number of dinucleotide repeats at a given locus include use of PCR to amplify the regions in question. One uses primers to locate and initiate amplification of a particular loci in a sample. After the amplification, one determines the particular alleles at a given locus in the sample by determining the fragment length of the amplified material. By determining the fragment length, one can determine the number of dinucleotide repeats at that location. Thus, the particular allele at that locus is identified.

Artifacts, however, can be created in the process, which may render difficult accurate determination of the actual allele at a given locus. These artifacts may be a result of PCR stutter, which can result from mistakes in amplification of the repeated nucleotides in the region being studied. Specifically, the polymerase in the PCR reaction may slip and miss one or more of the repeat units that are present in the studied nucleic acid region. In addition, an extra A nucleotide may be added during amplification. Thus, when PCR stutter and/or plus A distortion occurs, the amplification products typically will include not only the correct amplified allele, but also shorter repeats missing one or more of the repeat units of the allele. In fact, the data may show multiple peaks of various lengths where the data should reflect only one length.

It would also be useful to provide improvements at various stages of processes for determining alleles to increase the level of accuracy and confidence placed in given allele results obtained from generated data.

### SUMMARY OF THE INVENTION

Certain embodiments of the invention provide a computer-implemented method for making allele calls. In certain embodiments, the method comprises:

receiving data representing nucleic acid information;

applying at least two different allele calling algorithms to the data to provide a result for each algorithm; and

depending on agreement between the results of each algorithm, identifying an allele call within the data and assigning a confidence level for each call.

Certain embodiments of the invention provide a computer-implemented method for obtaining an allele call report, comprising:

receiving data representing nucleic acid information;

applying at least two different algorithms to the data to provide an allele call report;

generating a first algorithm quality value based on one of the at least two different algorithms;

generating a second algorithm quality value based on another of the at least two different algorithms;

generating an allele call report quality value based on at least the first and second algorithm quality values; and

predicting the accuracy of allele call report in view of the generated allele call report quality value.

According to certain embodiments of the invention, unique calling algorithms are also provided.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 depicts an overview block diagram for use with methods and systems consistent with certain embodiments of the present invention when providing allele calls.

FIG. 2 depicts a flow chart of the steps performed by a data processing system in processing allele calls when practicing methods and systems consistent with certain embodiments of the present invention.

FIGS. 3A-3D depict exemplary allele calling algorithms for use with methods and systems consistent with certain embodiments of the present invention.

FIG. 4 depicts a flow chart of the steps performed by the committee machine of FIG. 1 for use with methods and systems consistent with certain embodiments of the present invention.

FIG. 5 depicts a block diagram of a system for practicing methods and systems consistent with certain embodiments of the present invention.

FIG. 6 depicts data that may be generated and then interpreted using certain embodiments of the present invention.

FIG. 7 depicts data discussed in Example II (Envelope Caller).

FIG. 8 depicts data discussed in Example III (Optimizer Caller).

FIG. 9 depicts methods for searching for an allele that is discussed in Example III (Optimizer Caller).

FIGS. 10 through 12 depict data that can be evaluated with the heuristic algorithm according to certain embodiments.

FIG. 13 illustrates a typical standard heterozygous allele signature. (Circles denote user annotated allele calls. x-axis is in base pairs. y-axis is in A/D counts (voltage intensity))

FIG. 14 illustrates steps in the allele calling routine according to the embodiments discussed in Example V (Committee Machine Processing). First the signal is simplified via sampling and its peaks are located. This forms the target signal that is to be approximated. The two interconnected boxes indicate the process of varying the parameters and testing how closely the resulting signal matches the sampled version of the original. The set of parameters that yield the closest match contain the allele calls.

FIG. 15 depicts data discussed in Example V (Committee Machine Processing). It illustrates hypothesis formation in the optimizer routine. The two columns represent the optimal solution (left column) and a suboptimal solution (right column). Panel (a) shows the target vector with the two red lines showing the location of the candidate peaks. Panel (c) shows the hypothesis formed using different values of stutter and ⁺A. Panel (c) shows the residual error resulting from subtraction of the signal in panel (c) from the signal in panel (a) (sum squared error=0.0355). Panels (b,d,f) show the same process for a slightly different allele hypothesis. This is a poor hypothesis and the residual is rather significant (SSE=0.4715). The x-axis is somewhat meaningless at this point since it gets mapped back to base-pair indices after the winning hypothesis is chosen.

FIG. 16 depicts data discussed in Example V (Committee Machine Processing), and shows division of heterozygous signal into panels by the Envelope Caller algorithm. The panels are ranked according to signal energy and the three of interest are labeled p1, p2 and p3 with the two panels containing strong allele signatures being shaded in blue. Circles denote user annotated allele calls. (x-axis is in base pairs. y-axis is in A/D counts (voltage intensity))

FIG. 17 illustrates an example of how reporting could be accomplished as discussed in Example V (Committee Machine Processing). These are examples where consensus was not reached and show data that is difficult to interpret.

FIG. 18 depicts an overview block diagram of the system according to certain embodiments.

FIG. 19 depicts exemplary data of the effects of localvectorMin on baselining when the signal contains no “structure” (“structure” is “useful information” such as peaks.

FIG. 20 depicts exemplary data according to certain embodiments where the positive structure is eliminated.

FIG. 21 depicts an exemplary bottom baseline after eliminating the negative spikes.

FIG. 22 depicts exemplary data according to certain embodiments where baselining is generated by averaging the top and bottom.

FIG. 23 depicts the baselined signal according to certain embodiments.

FIG. 24 depicts exemplary data according to certain embodiments.

FIG. 25 depicts exemplary data showing detail of the peak location according to certain embodiments.

FIG. 26 depicts exemplary data when the peak is symmetric.

FIG. 27 depicts exemplary data when the peak is not symmetric.

FIG. 28 depicts exemplary data when the peak is not symmetric.

FIG. 29 magnifies the region marked in red in FIG. 28.

FIG. 30 depicts exemplary data by calculating the first derivative by fitting polynomials according to certain embodiments.

FIG. 31 depicts exemplary data using k to smooth the derivative according to certain embodiments.

FIG. 32 depicts peaks in certain exemplary data.

FIG. 33 depicts peaks in certain exemplary data.

FIG. 34 illustrates how, according to certain embodiments, to avoid certain artifacts.

FIG. 35 illustrates exemplary data showing a peak with shoulders.

FIG. 36 illustrates exemplary data which shows how, in certain embodiments, one may find a shoulder by analyzing the second derivative.

FIG. 37 illustrates exemplary data which shows how, in certain embodiments, one may find a shoulder by analyzing the second derivative.

FIG. 38 illustrates the final result of the peak detector's shoulder detections according to certain embodiments.

FIG. 39 depicts exemplary data of peaks, sizes, and a matching.

FIG. 40 illustrates a mesh of execution times according to certain embodiments.

FIG. 41 illustrates how each curve may hold constant the number of extra peaks according to certain embodiments.

FIG. 42 illustrates how each curve may hold constant the number of sizes in the size-standard definition according to certain embodiments.

FIG. 43 depicts linear interpolation according to certain embodiments.

FIG. 44 illustrates linear interpolation according to certain embodiments.

FIG. 45 illustrates exemplary data of a size calling algorithm according to certain embodiments.

FIG. 46 depicts a flow chart of the system according to certain embodiments.

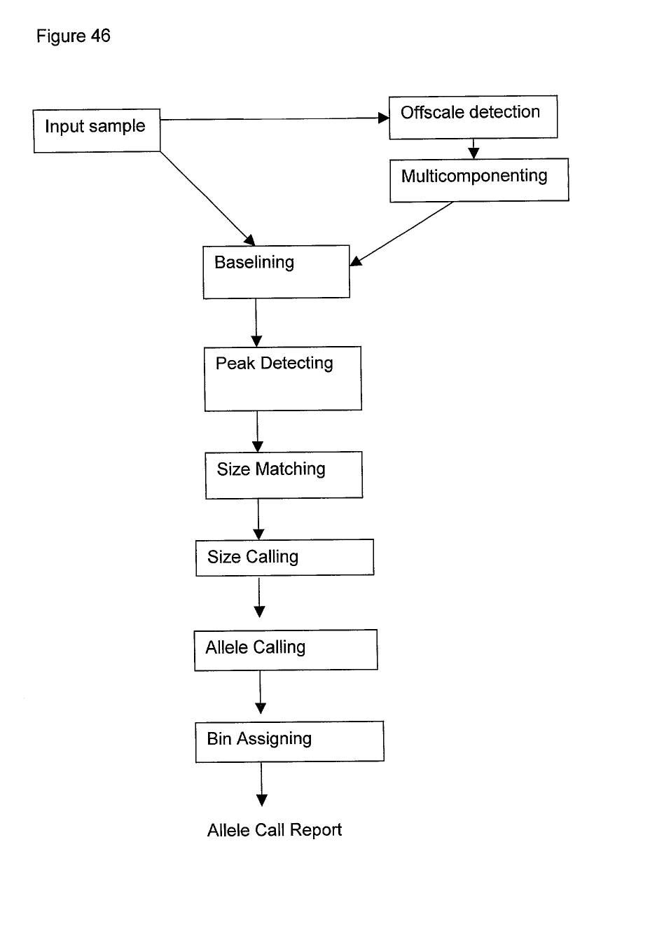

According to certain embodiments, the system may contain one or more of the algorithms depicted in FIG. 46, which result in an allele call report.

### DETAILED DESCRIPTION

The following detailed description of the invention refers to the accompanying drawings. Although the description includes exemplary implementations, other implementations are possible, and changes may be made to the implementations described without departing from the spirit and scope of the invention. The following detailed description does not limit the invention. Instead, the scope of the invention is defined by the appended claims. Wherever possible, the same reference numbers will be used throughout the drawings and the following description to refer to the same or like parts. Several documents are discussed throughout this application. All of those documents are expressly incorporated by reference herein in their entirety for any purpose. Patent Cooperation Treaty Application No. ______ (not yet assigned), filed Jul. 23, 2001, naming as inventors Heinz Breu and Hugh J. Pasika, naming as applicant Applera Corporation, and titled “Methods and Systems for Evaluating a Matching Between Measured Data and Standards” is incorporated by reference for any purpose.

The following definitions are provided for terms used in this application.

Allele—An allele is one of two or more alternate forms at the same locus. With respect to a given locus, a diploid organism may be homozygous (having the same allele on each of the two homologous chromosomes) or heterozygous (having a different allele on each of the two homologous chromosomes). Non-diploid organisms may have more than two alleles.

Allele Calling—When fragment analysis is performed, the region of nucleic acid containing the marker is flanked by known primer sites which permit localization of the allele. For example, changes in the allele may result in different fragment lengths. Thus, for these alleles, determination of the length of the nucleic acid sequence between primers is referred to as allele calling. For example, if two alleles are present, there will be two pieces of nucleic acid with different lengths.

Locus—A unique chromosomal location defining the position of an individual nucleic acid sequence.

Allele Signature—During PCR amplification, PCR stutter often occurs, which results in additional peaks that emerge in a predictable pattern. Another artifact that may appear is plus A distortion. The combination of the original signal, the stutter, and other artifacts is referred to as the allele signature.

Marker—Markers can be thought of as landmarks in the genome and can appear in noncoding regions of nucleic acid. Their use in linkage mapping stems from their polymorphism. Many different types of markers exist.

Algorithm—An algorithm is a process of one or more steps for accomplishing a result. The word “component” is used interchangeably in this application with the word “algorithm.”

Unless specifically indicated otherwise, use of a singular term in this application encompasses the plural as well. For example, use of the term “an algorithm” encompasses at least one algorithm, but may include more than one algorithm.

SYSTEM

According to certain embodiments, the system includes one or more of the algorithms or components depicted in the flowchart shown in FIG. 46. The following sections discuss each of the algorithms set forth in that flowchart. The system in certain embodiments will include all of the algorithms in FIG. 46. In certain embodiments, the system will not include all of those algorithms. In certain embodiments, the system may obtain information that has already been subjected to one or more prior algorithms set forth in FIG. 46 and then proceeds with one or more of the subsequent algorithms set forth in FIG. 46. For example, the system may start with information that has already been subjected to an offscale and multicomponenting process or similar processes, and then proceeds with one or more of the subsequent algorithms shown in the flowchart. In certain embodiments, the system may provide information obtained from algorithms to another system that then uses that information to obtain a result.

In certain embodiments, the system allows automated scoring or sizing of DNA fragments. In certain embodiments, these fragments are mostly Microsatellites but other markers can be used (e.g. amelogenlin, snp markers). The scores from these markers can be used in a variety ofapplications. Two exemplary, but not limiting, applications for certain embodiments of the system are Linkage Mapping and Databasing for Human Identification (HID).

In certain embodiments of Linkage Mapping, the allele calls from a number of samples of related individuals are used to define a region of DNA in which a gene of interest lies.

In certain embodiments of human identification (HID), the allele calls for a set of markers form a profile for an individual. This can be stored in a database and compared to the profiles obtained from crime scenes to match a suspect to a crime. The profile of an individual may also be used for determining paternity.

The following description of algorithms and processes that may be used in certain embodiments consistent with the present invention includes discussion of specific algorithms that may be applied to obtain a particular desired result. For convenience, specific nomenclature has been selected to refer to these algorithms. Systems and methods consistent with the present invention, however, are not limited to the disclosed algorithms. They may include other algorithms that provide the same or similar results.

OFFSCALE DETECTION

In certain embodiments, the system includes an offscale detection algorithm. If the data (e.g., a fluorescent signal) in any filter for a certain scan number is larger than a set maximum, an offscale detection algorithm will treat that position (scan number) as offscaled. Thus, that data for that scan number is flagged. In certain embodiments, offscale detection is performed in the data collection process. In such embodiments and in certain other embodiments, the system need not perform offscale detection.

MULTICOMPONENTING

In certain embodiments, the system includes a multicomponenting component for sample files. A multicomponenting algorithm is a process that converts optically filtered data to day concentration data. For example, the raw data may include fluorescence of different colored dyes that overlap. The multicomponenting purifies such signals such that a signal from a different dye does not interfere with the signal from each other dye. In certain embodiments, the multicomponenting process takes the matrix values read in from the sample files and multiplies them to the raw signal to get the multicomponented signal data.

For example, in certain embodiments, the raw data signal F is a list of ƒ-tuples that give the response from each of the f optical filters used by the instrument. The information is converted into a list D of d-tuples that give the concentration of each dye. To do so, the system is given a chemometric matrix M where D=FM. The system, therefore, simply multiplies the vector of filter responses by the chemometric matrix.

In certain embodiments, multicomponenting is performed in the data collection process. In such embodiments and in certain other embodiments, the system need not perform multicomponenting.

BASELINING

In certain embodiments, the system includes a baselining algorithm, which subtracts out certain baseline shifts from the signal. In certain embodiments, baseline shift may be caused by inconsistent operating conditions, such as temperature fluctuation or differences in loading conditions. For example, when using capillaries, baseline shift may occur with different pressures or volumes when loading the capillaries.

In certain embodiments, the baselining algorithm employs three parameters: window size, smooth size and spike size. In certain embodiments, the system fixes the smooth size to −1 (no smoothing) and spike size to 21. In certain embodiments, the system uses different window sizes for different instruments. For example for Applied Biosystems 310 and 377 instruments, the system uses 99 for the window size and for Applied Biosystems 3700 instrument, the system uses 251 for the window size.

In certain embodiments, the baselining algorithm finds a bottom baseline that rides under the noise, and a top baseline that rides over the noise. It then averages the two.

In certain embodiments, the baselining algorithm works by finding minima and maxima in a signal. In certain embodiments, the baselining component defines localVectorMax to be the maximum signal value in a window of size k=2k₂+1 about a point x:

localVectorMax(signal,x,k)=max{signal(i): x−k₂≦i≦x+k₂}.

The parameter k is called the “Baseline Window Size”. Similarly, the baselining component defines localVectorMin to be the minimum signal value in a window of size 2k₂+1 about x:

localVectorMin(signal,x,k)=min{signal(i): x−k₂≦i≦x+k₂}.

In certain embodiments, these operators are overloaded to provide vectors of minima and maxima:

localVectorMin(signal,k)=[localVectorMin(signal,x,k):x=1,2, . . . , n],

localVectorMax(signal,k)=[localVectorMax(signal,x,k):x=1,2, . . . , n],

In certain embodiments, to baseline a signal, one eliminates the “useful information” like fragment peaks, in the signal. For example, assume that the structure will not extend over k=101 units, say. Then the baseline in effect at a given point should be within this window.

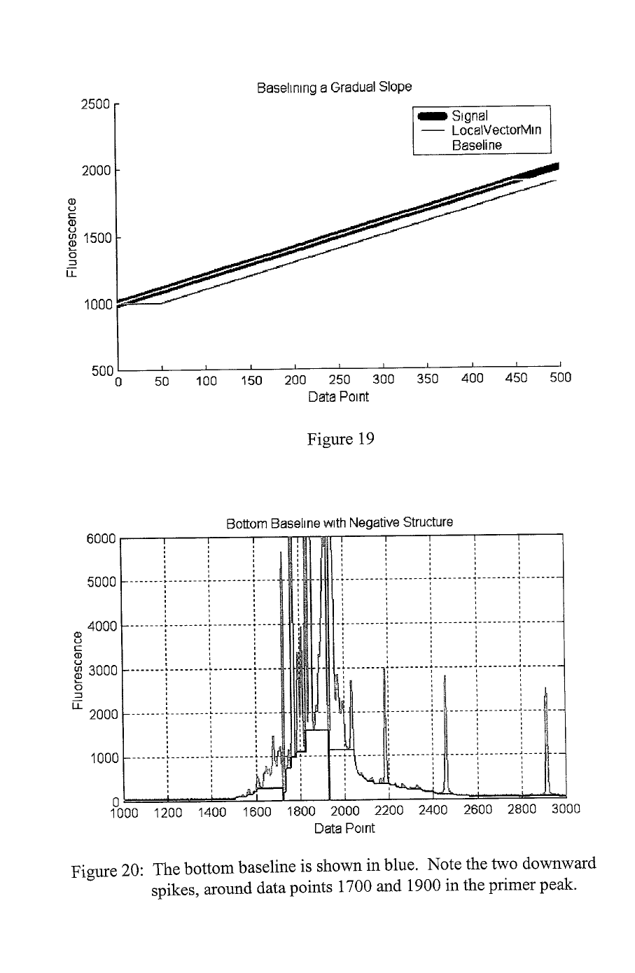

An example in practice according to certain embodiments, is shown in FIGS. 19 through 23. In FIG. 19, the signal contains no structure, but has a constantly sloping baseline. In certain embodiments, the baselining algorithm should leave the signal largely untouched. But consider the effect of localVectorMin in the figure. It took too much from the signal.

The positive structure can be eliminated by executing

bottom=localVectorMax(localVectorMin(signal,k),k);

as shown in FIG. 20. The resulting bottom baseline, shown in blue, still retains some negative structure. In certain embodiments, such structure should not extend over any significant distance at all, and so can be eliminated with a narrower window, say of size σ=21 (i.e., the spike size).

bottom=localVectorMin(localVectorMax(bottom,σ), σ);

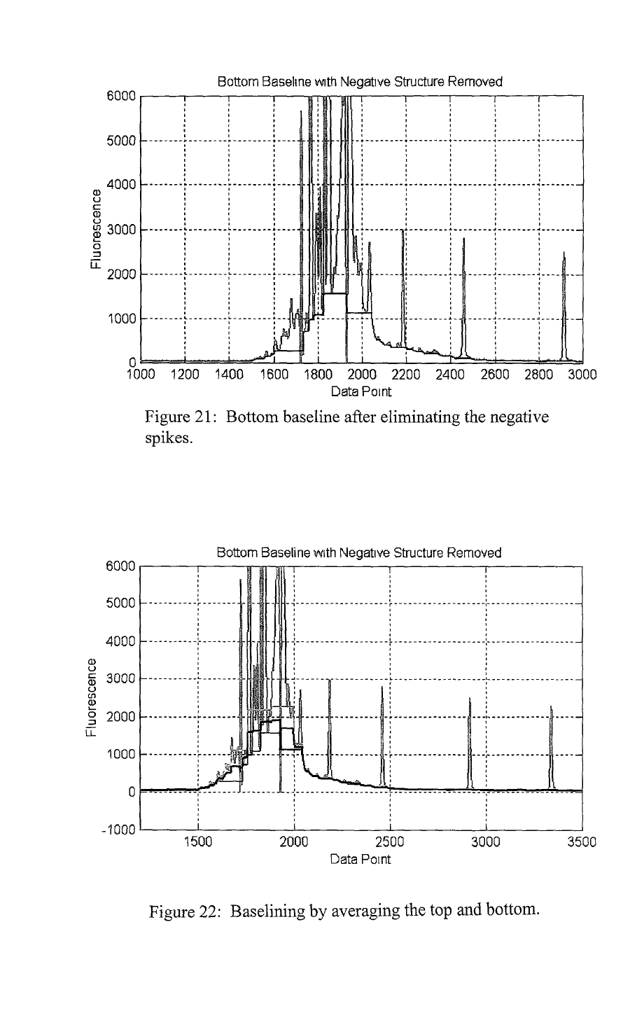

The result is shown in blue in FIG. 21.

If one wants a baseline that goes through the “middle” of the background noise, in certain embodiments, one can compute a top baseline and average the two. In certain embodiments, to compute the top baseline, one eliminates negative spikes first, and then eliminates the positive peaks:

top=localVectorMin(localVectorMax(signal,σ), σ);

top=localVectorMax(localVectorMin(top, k), k);

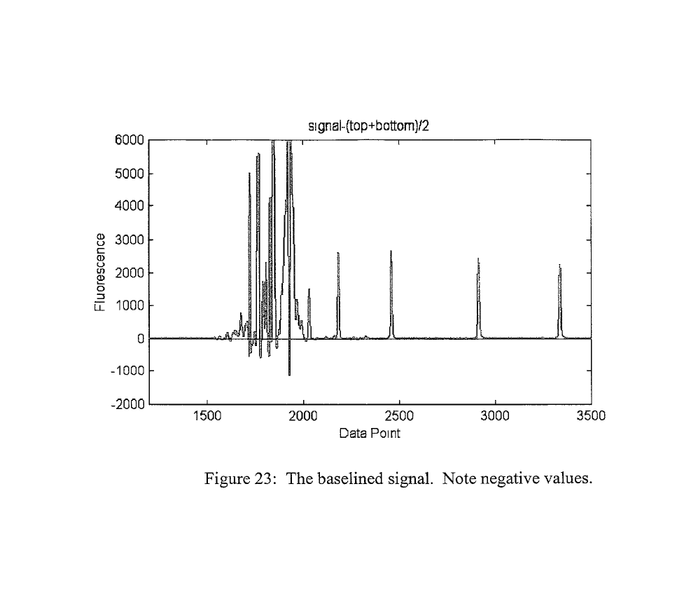

FIG. 22 shows the top baseline in green, the bottom baseline in blue, and the average baseline in black. It is a simple matter for the system to remove the baseline by subtracting it from the signal, as shown in FIG. 23.

In certain embodiments, the baselining window size is user-settable. In certain embodiments, one skilled in the art will be able to get an appropriate window size. In certain embodiments, windows that are too small will track peaks too closely, so that the baselined peaks will appear short. In certain embodiments, windows that are too large will not track baseline variations, such as the primer peak tail, closely enough, so that the baselined peaks will appear high and under-resolved.

PEAK DETECTION

In certain embodiments, the system uses a peak detection algorithm. Such an algorithm helps predict where in the generated data there are actual peaks. In certain embodiments, such an algorithm employs four parameters: degree, window width, tauB (smallest slope at which peaks start) and tauE (smallest slope at which peaks end). In certain embodiments, the system uses 3 for degree, 99 for window width, 0.0 for tauB and 0.0 for tauE. In certain embodiments, the system uses degree 2.

In certain embodiments, the algorithm also takes two additional parameters: min peak height and min peak width (full width at half maximum). In certain embodiments, the system uses these two additional parameters to filter out the noise peaks. In such embodiments, peaks whose height is lower than min peak height or whose full width at half maximum is less than min peak width are tossed out in a filtering process. In certain embodiments, the system fixes the min peak width at 2 (scan numbers). For the min peak height, in certain embodiments, the system provides two choices: auto-determined and user specified. In the auto-determined mode, in certain embodiments, the system uses a baselining algorithm to figure out the noise level and the min peak height is picked as 10 times that noise level. In certain embodiments, one may use the particular baselining algorithm discussed above. In the user specified mode, in certain embodiments, the user specifies the min peak heights for blue/green/yellow/red/orange dyes.

One skilled in the art will be able to determine suitable degree and window width, which in certain embodiments is related to the data generated by the specific instrument employed.

In certain embodiments, the Size-calling peak detector is called the Savitzky-Golay detector.

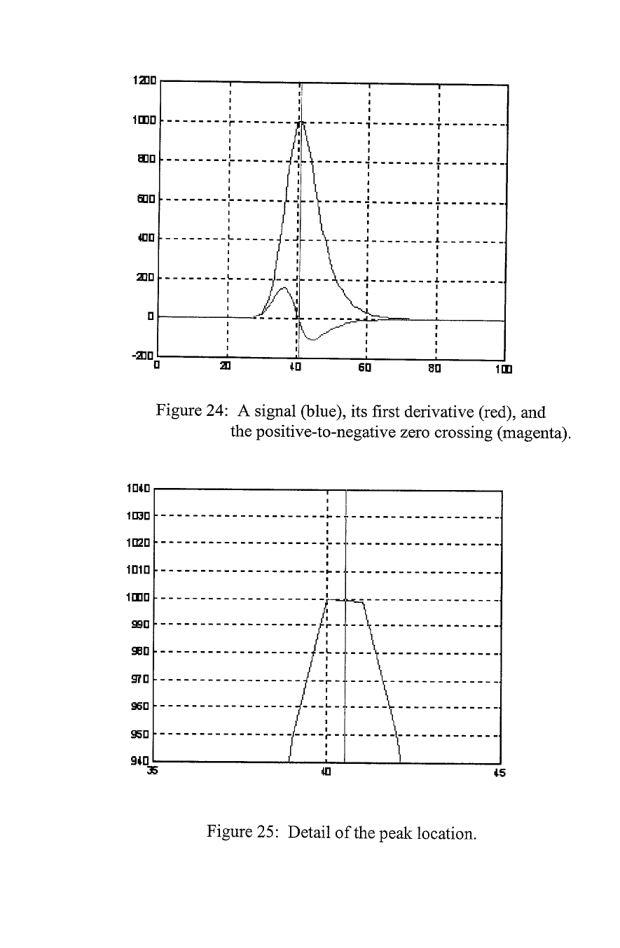

A peak is a local maximum in a signal. The peak detector detects a peak when it sees a positive-to negative zero crossing in the first derivative. FIG. 24 shows an example. Note that this position is different from the highest point on the peak (due to the calculation of the first derivative), as shown in FIG. 25. In certain embodiments, one may use the zero crossing as the peak position and in certain embodiments one may use the highest point as the peak position.

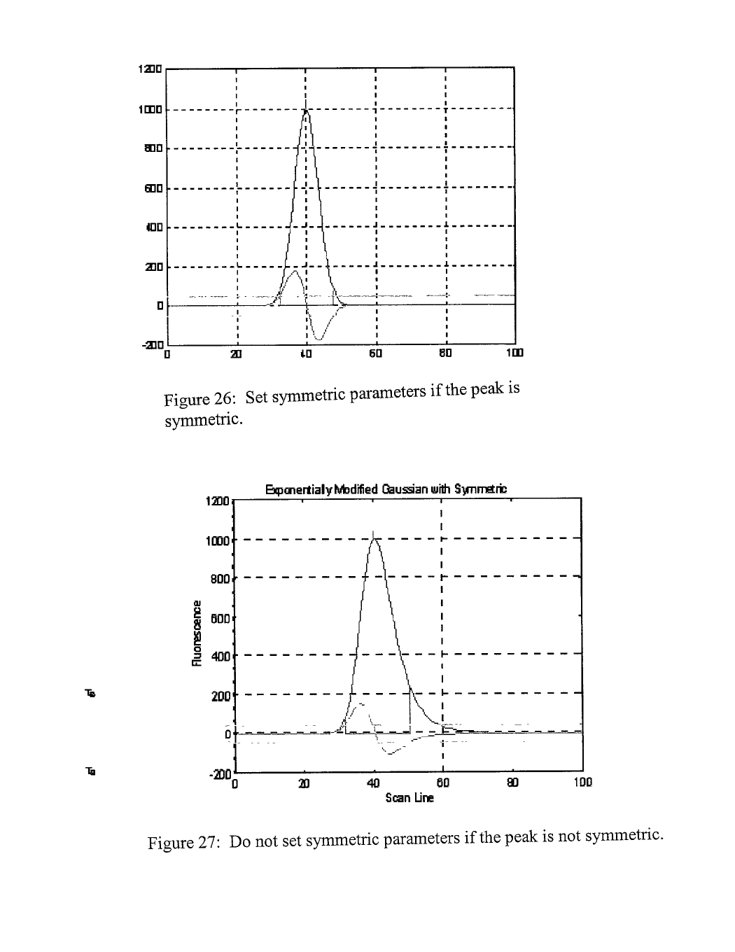

The Savitzky-Golay detector estimates the beginning and the end of a peak by thresholding the rising edges of the first derivative using two user-specified parameters, a non-negative TB and a non-positive Γ_{E}. In certain embodiments, Γ_{B} is called the “Slope Threshold for Peak Start”, and Γ_{E} is called the ‘Slope Threshold for Peak End”. The detector finds the start of a peak by searching to the left of the peak position. The peak starts where the first derivative crosses Γ_{B} from negative to positive. The detector finds the end of a peak by searching to the right of the peak position. The peak ends where the first derivative crosses Γ_{E}, also from negative to positive. If the peak is symmetric (a Gaussian, for example), typically |Γ_{B}|=|Γ_{E}|, as illustrated in FIG. 26.

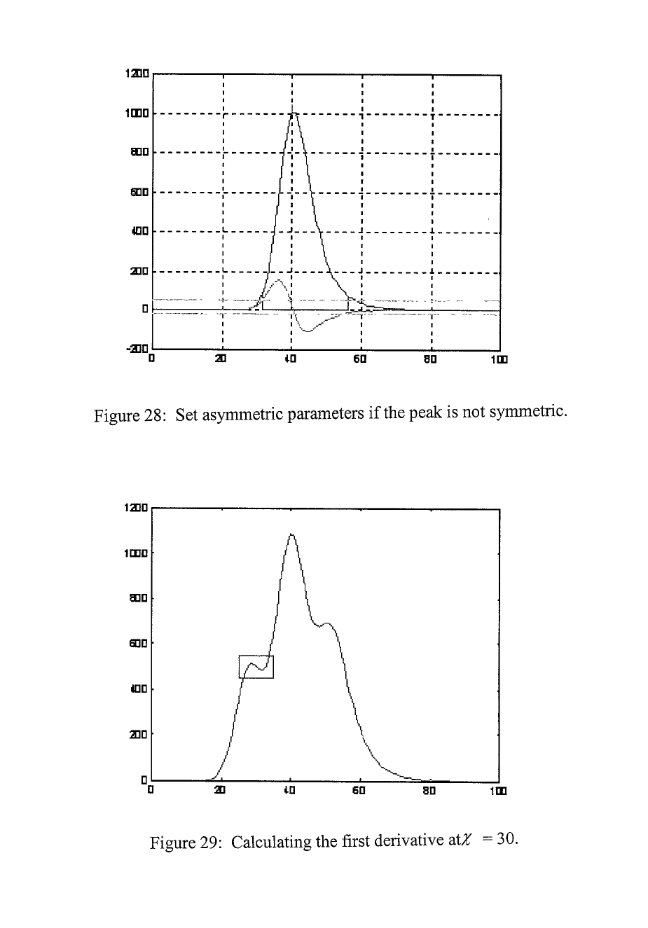

On the other hand, if the peak is asymmetric (an Exponentially Modified Gaussian, for example), then setting symmetric start and end conditions may give strange results, as shown in FIG. 27. In this case, typically one would set asymmetric termination criteria, as shown in FIG. 28. In certain embodiments, however, it may suffice simply to set Γ_{B}=Γ_{E}=0, since the background noise may not permit finer values.

The peak detector calculates the first derivative with Savitzky-Golay “windows” of width k as follows. Suppose one wants the first derivative at x=30 in FIG. 28. FIG. 29 magnifies the region marked in red. The algorithm first fits a polynomial curve to the k data.

For example, the red curve is a quadratic fit to the 5 points, and the green curve is a cubic fit. The algorithm then differentiates the curve, and evaluates the derivative at x=30. Note that, in this case, the first derivative from the quadratic is nearly 0 at x=30, while the first derivative from the cubic more closely approximates the underlying signal.

The Savitzky-Golay technique may compute this derivative without having to fit a curve to every window (W. H. Press, B. P. Flannery, S. A. Teukolsky, and W. T. Vetterling, General linear least squares, In Numerical Recipes in C, chapter 14.3, pages 528-539, Cambridge University Press, 1988.). The parameter d, called “Polynomial Degree” in certain embodiments, determines the degree of the polynomial to use.

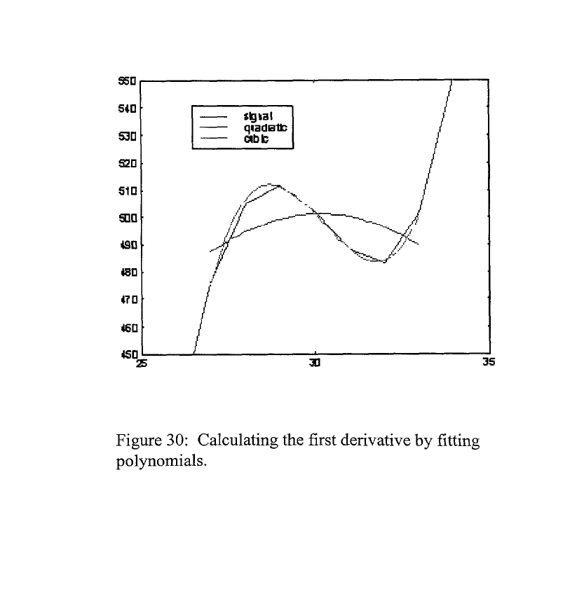

In certain embodiments, one uses the quadratic (d=2) in a small special-case application. In certain embodiments, one uses the cubic (d=3), since it follows small “rider” peaks quite well, as FIG. 30 illustrates. In certain embodiments, one uses d=4.

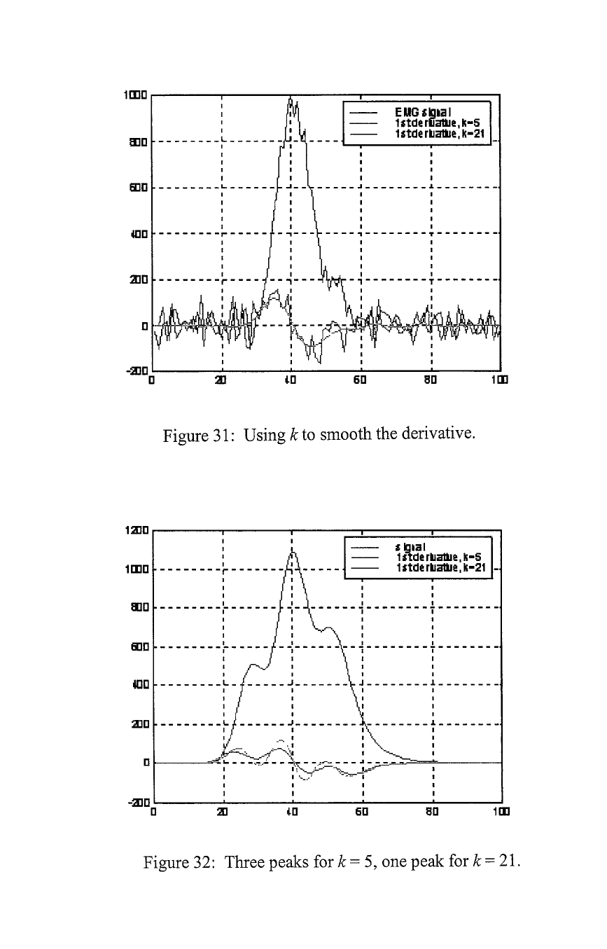

The window size k is a control parameter for the detector. In certain embodiments, one sets k to 1.5 times the expected (not minimum) full peak width at half-max (FWHM). The effect of k may be evident in the presence of noise. FIG. 31 shows the first derivative calculated with k=5 as a red curve, and with k=21 as a green curve. In certain embodiments, the Savitzky-Golay technique is a kind of smoothing, with larger values for k resulting in smoother curves. In certain embodiments, the Savitzky-Golay technique will not force peaks down (by lowering the maximum) and out (by raising the edges), in contrast with smoothing by averaging.

In certain embodiments, although large values for k effectively track isolated peaks, they can swamp peaks that are not fully resolved. In FIG. 32, the algorithm would detect three peaks for k=5, but only one for k=21.

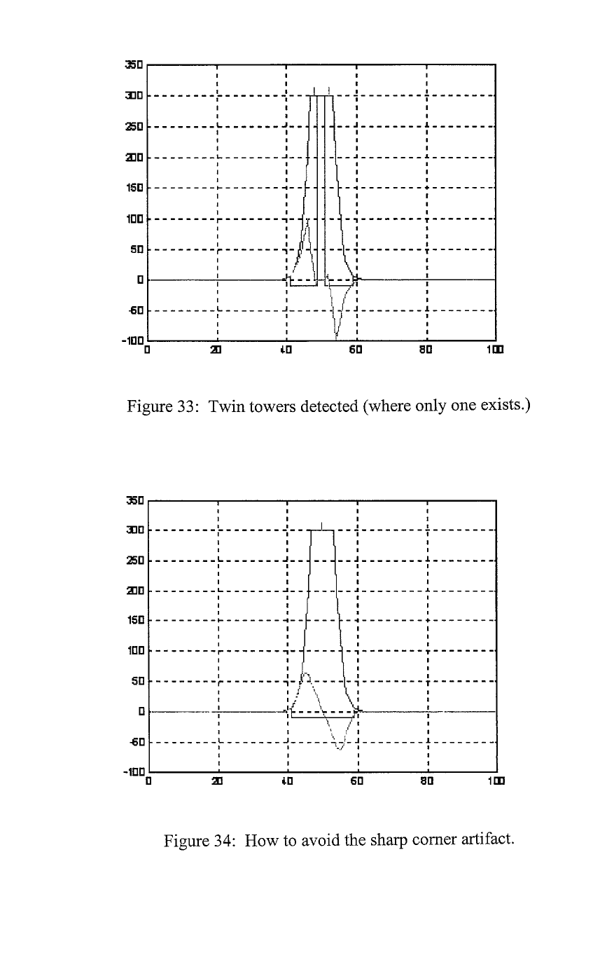

In certain embodiments, sharp comers can cause artifacts in the algorithm. The truncated curve in FIG. 33 should be seen as a single peak. However, one can see spurious zero crossings with d=3 and k=5 (here Γ_{B}=5 and Γ_{B}=−5).

To avoid these artifacts, in certain embodiments, one sets k larger than the FWHM of the feature one wishes to detect. For example, FIG. 34 shows the effect of k=11.

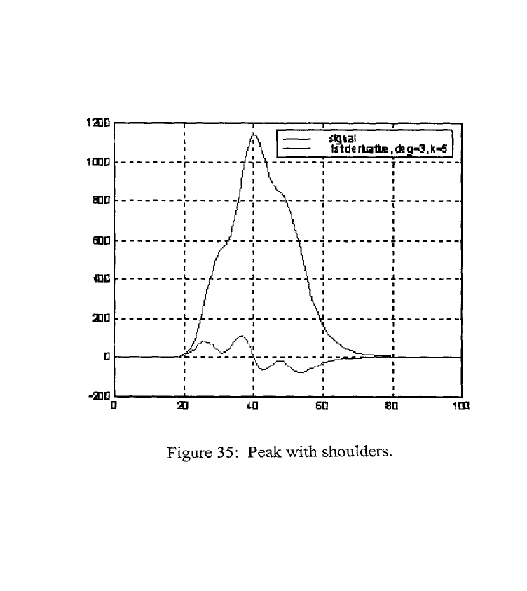

Except for the sharp corner artifact, in certain embodiments, the Savitzky-Golay detector will detect multiple peaks only if clear valleys separate them. For example, in such embodiments, in FIG. 35, the detector will detect only one peak.

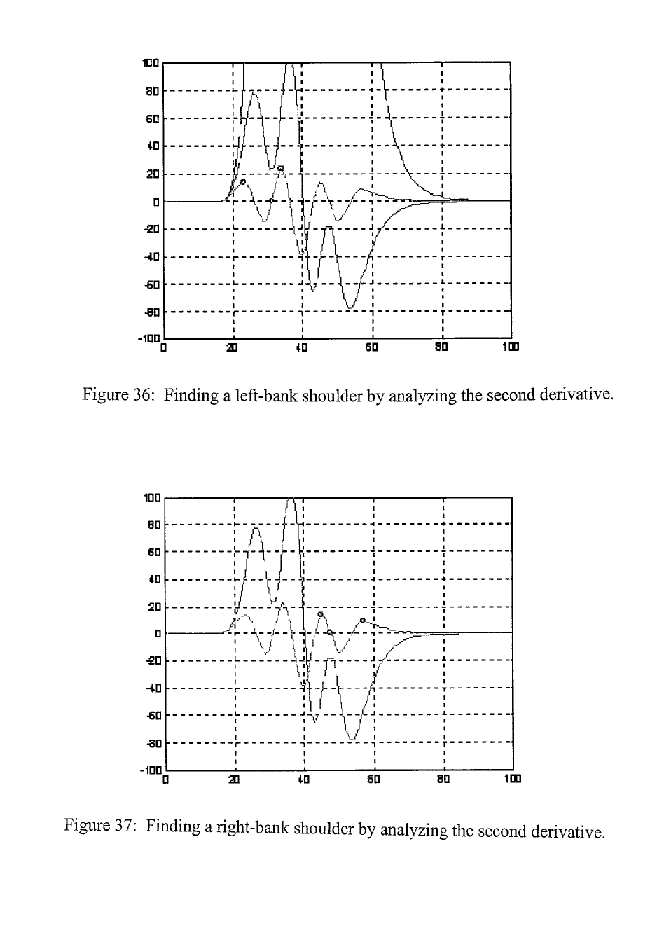

This peak does, however, has shoulders. In certain embodiments, one may have the peak detector find shoulders by examining the second derivative. In certain embodiments, the algorithm detects left- and right-bank shoulders differently, though similarly. For a left-bank shoulder, the first derivative is positive and is “trying” to cross zero (thereby causing a peak). So the position of the shoulder is marked by a local minimum in a positive first derivative. The algorithm finds this location by looking for a negative-to-positive zero crossing of the second derivative. The beginning of the shoulder is the point at which the slope stops increasing so quickly (in preparation for the shoulder), that is by a local maximum in the second derivative. The end of the shoulder is marked by the same condition (in preparation for the peak or another shoulder). FIG. 36 marks these three locations (start, position, and end of shoulder) with small circles.

For a right-bank shoulder, the first derivative is negative and is “trying” to cross zero (thereby causing a peak). So the position of the shoulder is marked by a local maximum in a negative first derivative. The algorithm finds this location by looking for a positive-to-negative zero crossing of the second derivative. Again, the beginning and end of the shoulder are marked by local maxima in the second derivative. FIG. 37 marks these three locations (start, position, and end of shoulder) with small circles.

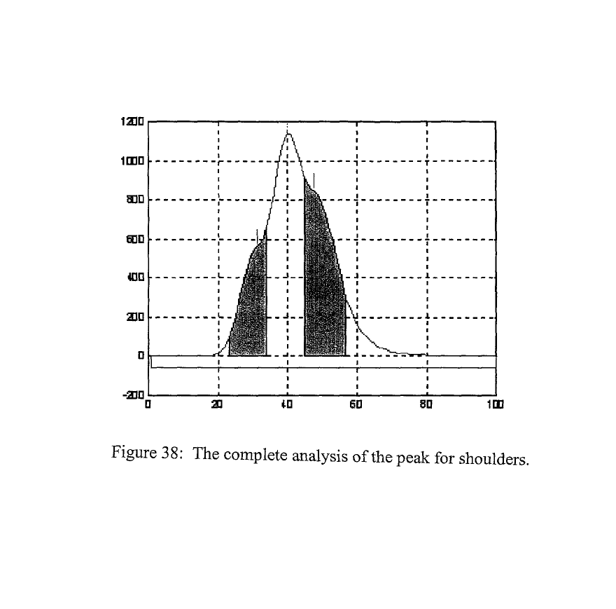

The plot in FIG. 38 shows the final result of the peak detector's shoulder detection according to certain embodiments.

In certain embodiments, once the peak detector has found all peaks to within the resolution of the first derivative, it selects only those peaks that meet user-defined minimum height and width constraints. The height of a peak is the maximum signal value from its start to its end. In certain embodiments, the peak detecting algorithm will report a peak only if the peak's height is at least that of the peak amplitude threshold for that dye. In certain embodiments, the thresholds for the blue, green, yellow, red, and orange dyes are called respectively “B:”, “G:”, “Y:”, and “R:”, and “0.”

In certain embodiments, the peak detecting algorithm will report a peak only if the peak's width is at least that of the peak width threshold. In certain embodiments, this threshold is the same for all dyes.

Peak Area

Once detected, the peak detecting algorithm measures the area of a peak to be sum of the (baselined) fluorescence values from the start of the peak to its end. Note that this may result in a negative area if more of the peak is below the baseline than above it. In certain embodiments, one may smooth the baseline using an endpoint smoothing (averaging).

In certain embodiments, those skilled in the art will be able to estimate the peak width and detection threshold for the peak detector.

SIZE STANDARD MATCHING

Certain embodiments employ a size standard matching algorithm (which may also be referred to as a “size standard matcher” or “size matcher”). Such an algorithm matches data generated with a standard sample to actual sizes that should exist in the standard sample. For example, one may use a standard sample with nucleotide lengths 110, 114, 117, 120, and 125. One runs the standard sample and obtains several data peaks. The size standard matching algorithm predicts the peaks that correspond to the five known nucleotide lengths. Thus, one can subsequently compare data in a sample to those peaks to determine the nucleotide lengths of fragments in a sample.

In certain embodiments, a size standard matching algorithm includes three parameters: ratio factor (the importance of peak height vs the importance of the local linearity), min acceptable quality (used for ending dynamic programming iteration), and number of extra peaks (the number of peaks participated in size matching is the number of size standard definition fragments plus the number of extra peaks). In certain embodiments, the algorithm fixes the ratio factor to 0.6 and min acceptable quality to 0.75. In certain embodiments, the algorithm fixes the number of extra peaks to 10 for Applied Biosystems 310/377 instrument data and 25 for Applied Biosystems 3700 instrument data.

In certain embodiments, a statistically based quality value is generated for the matching result.

In certain embodiments, one skilled in the art will be able to adjust the number of extra peaks that may be used with a given instrument.

In certain embodiments, the algorithm ignores the peaks located within the offscale regions in the sample. In certain embodiment, the algorithm fails the size matching process if the size standard definitions are not fully matched in the matching process.

In certain embodiments, the algorithm implements two primer peak detection methods. The first is the primer-peak-height-supression method. This method replaces the peak heights of the highest peaks with the peak height of the middle peak, assuming that the primer peaks are among the highest. The second is to find the primer peak location. The method assumes that the primer peak locates within the first half of the signal and the size standard fragments locate in the second half of the signal. For example, one takes the mean peak height of all the peaks in the second half and multiples that mean by five to get the potential primer peak height. The method works backwards in the first half of the signal to find the last primer peak.

In certain embodiments, a size-standard matching algorithm takes as input a list of peaks (e.g., from an electropherogram) and a list of fragment sizes (e.g., in nucleotides). It produces as output a matching, that is, a list of pairs of the form <peak,size>, where each peak and each fragment size appears at most once. In certain embodiments, a size standard matching algorithm evaluates a matching, and uses an algorithm for finding good matchings.

Certain embodiments employ an algorithm that evaluates a matching by treating its two constituent sequences as sequences of edges between points. A matching is also a correspondence between edges. Two edges, e₁ and e₂, that share an endpoint define a ratio of lengths r=|e₂|/|e₁|. Again, a matching is also a correspondence between ratios. Under the assumption that the relation between peak position and fragment size is “more or less” linear, corresponding ratios typically should be equal. In certain embodiments, the algorithm derives a ratio cost to measure this property. In certain embodiments, the component also concentrates on big peaks by deriving a height cost. The total cost of a matching is a weighted sum of these constituent costs.

In certain embodiments, the algorithm formulates the size standard matching problem as finding a matching with maximum cost. In such embodiments, the cost is separable. That is, with some additional mathematics, the algorithm can maximize subsequences independently. In certain embodiments, the cost also enjoys the advantage of being local, thereby compensating for global deviations from linearity. This cost also leads to a quality value between 0 and 1.

A size standard is a set of DNA fragments, each of known size. The definition of a size standard is simply a list of these sizes. Note that a size-standard definition typically does not depend on the instrument on which the size standard is run, and therefore not on any particular set of run conditions either.

An in-lane size standard is a set of peaks resulting from running a size standard on an instrument. One determines the positions and the heights of the peaks.

In certain embodiments, a size standard matching algorithm takes as input an in-lane size standard and a size standard definition. It produces as output a matching, that is, a list of pairs of the form (peak,size), where each peak and each fragment size appears at most once. A peak has a position (e.g., in scan numbers) and a height (e.g., in fluorescent units). Fragment sizes are given in nucleotides.

Assume that there are at least as many peaks as sizes. Furthermore, assume that every size has a corresponding peak, except possibly for some small number from the end of this list. This exception is meant to model the situation where a user may have stopped electrophoresis early, before the larger fragments have had a chance to elute.

In certain embodiments, one employs the following. Let P=└p₀, p₁, . . . , pₙ_{ₚ}₋₁┘ be a list of nₚ peak locations, given by increasing scan number, for example. Let H=└h₀, h₁ . . . , hₙ_{ₚ}₋₁┘ be a list of the corresponding np peak heights, given in fluorescent units, for example. The size standard definition S=└s₀, s₁, . . . , sₙ_{ₛ}₋₁┘ is a list of nₛ fragment sizes in increasing nucleotides. By assumption, nₚ≧nₛ. A size-standard matching is a set of pairs M={(i₀,0), (i₁,1), . . . , (iₙ_{ₛ},nₛ)} where the iⱼ are in increasing order, that is, where subscript j<k implies iⱼ<iₖ.

### EXAMPLE 1

### Peaks, Sizes, and a Matching

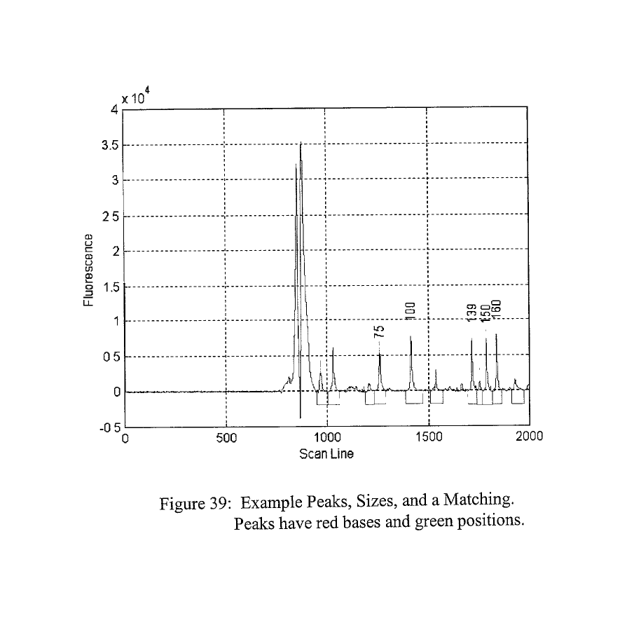

Consider the peaks, sizes, and matching displayed in FIG. 39. The list P contains nₚ=11 peak positions:

P=[968, 1029, 1203, 1259, 1412, 1535, 1714, 1751, 1785, 1837, 1928].

These nₚ peaks have heights H:

H=[2722, 6219, 1060, 5380, 7726, 1082, 7424, 1263, 7335, 7937, 1562].

The size standard definition has nₛ=5 sizes S:

S=[75, 100,139, 150, 160].

Finally, M is the matching shown in the figure:

M={(3, 0), (4, 1), (6, 2), (8, 3), (9, 4)}.

Big-Oh notation is used to express algorithm complexity. This notation is ubiquitous in worst-case and average-case resource analysis. Briefly, a function ƒ is said to be on the order of another function g (writtenƒ(x)=O(g(x)) if there exists positive constants c and N such that |ƒ(x)|≦c|g(x)| for all x≧N.

Evaluating a Matching

Assume there is a matching. In certain embodiments, the size standard matching algorithm evaluates a matching by examining its two constituent sequences. It treats the sequence of peaks as a sequence of edges between peaks, and similarly for the sizes. For example, M={(3, 0), (4, 1), (6, 2), (8, 3), (9, 4)} is the matching from Example 1. Its peak (index) sequence is [3, 4, 6, 8, 9], which has four edges, (3, 4), (4, 6), (6, 8), and (8, 9). Similarly, its fragment size definition (index) sequence is [0, 1, 2, 3, 4], which also has four edges: (0, 1), (1, 2), (2, 3), and (3, 4).

A matching is also a correspondence between edges. In this example, peak edge (6, 8) corresponds to definition edge (2, 3). Assume that two edges are adjacent if they share an endpoint. In this example, (4, 6) and (6, 8) are adjacent since they share peak 6. Two adjacent edges (i,j) and (j,k) define a ratio rᵢⱼₖ of lengths:

$${r}_{ijk} = \frac{{p}_{k} - {p}_{j}}{{p}_{j} - {p}_{i}} . \qquad ( 1 )$$

In certain embodiments, one can employ a more economical notation for size ratios for matching all sizes:

$${r}_{f} = \frac{{s}_{f + 2} - {s}_{f + 1}}{{s}_{f + 1} - {s}_{f}} . \qquad ( 2 )$$

Again, a matching is also a correspondence between ratios. In this example, peak ratio r₆₈₉ corresponds to size ratio r₂.

Under the assumption that the relation between peak position and fragment size is “more or less” linear, corresponding ratios typically should be equal. More formally, assume that the fragment of size sᵢ occurs at position pᵢ. If there are coefficients a and b such that pᵢ=asᵢ+b for all i, then

$$\frac{{p}_{k} - {p}_{j}}{{p}_{j} - {p}_{i}} = \frac{( {as}_{k} + b ) - ( {as}_{j} + b )}{( {as}_{j} + b ) - ( {as}_{i} + b )} = \frac{a ( {s}_{k} - {s}_{j} )}{a ( {s}_{j} - {s}_{i} )} = \frac{{s}_{k} - {s}_{j}}{{s}_{j} - {s}_{i}} . \qquad ( 3 )$$

To measure the similarity of a corresponding pair of ratios rᵢⱼₖ and r_{ƒ}, one may define their ratio cost cᵣ(i,j, k, ƒ) to be

$${c}_{r} ( i , j , k , f ) = \frac{min ( {r}_{ijk} , {r}_{f} )}{max ( {r}_{ijk} , {r}_{f} )} . \qquad ( 4 )$$

Note that 0≦cᵢ(i,j,k,ƒ)≦1 for all 0≦i<j<k<nₚ and 0≦ƒ<nₛ−2. Note also that cₚ(i,j,k,ƒ)=1 indicates the ideal of equal ratios. The ratio cost of a matching is the sum of its individual costs.

In certain embodiments, one has the matchings concentrate on the big peaks. To this end, one may define the height cost cₕ(i) of a matched peak i to be its height divided by the maximum peak height ĥ. More formally,

$$\begin{matrix}\hat{h} = {max}_{0 \leq j < {n}_{p}} {h}_{j} \\ and & ( 5 ) \\ {c}_{h} ( i ) = \frac{{h}_{i}}{\hat{h}} . & ( 6 )\end{matrix}$$

Again, 0≦cₕ(i)≦1 for all peaks 0≦i<nₚ, and cₕ(i)=1, in certain embodiments, corresponds to the ideal of a maximally tall peak.

In order to combine these two types of costs, one may weight and sum them. Since there are only two costs, a single weighting parameter α, where 0≦α≦1, will suffice. The total cost c(M) of a matching M is the weighted sum:

$$c ( M ) = α {\sum }_{( i , f ) ∈ M \\ ( j , f + 1 ) ∈ M \\ ( k , f + 2 ) ∈ M} {c}_{r} ( i , j , k , f ) + ( 1 - α ) {\sum }_{( i , f ) ∈ M} {c}_{h} ( i ) . \qquad ( 7 )$$

One may now formulate the size standard matching problem as finding a matching with maximum cost. Note that the cost is local in the sense that each element of the summation depends on at most three adjacent points. In certain embodiments, this property allows the size standard matching algorithm to compensate for global deviations from linearity.

Quality Measures

If one divides the cost of a matching by the maximum possible cost over all matchings, one would have a number between 0 and 1 that indicates its quality. What is this maximum possible cost? Every pair of ratios in such a matching would contribute its maximum value, namely α×1. There are a total of n−2 ratio pairs. Similarly, every matched peak would be at the maximum height, so that all n matched peaks (one for each definition size) contribute (1−α)×1. The maximum possible cost c, therefore, is:

ĉ=(n−2)α+n(1−α)=n−2α (8)

The quality of a matching M is therefore given by c(M)/ĉ.

Other possible quality measures include the sum of just the ratio costs, and the worst ratio cost in the matching.

An Efficient Algorithm

In certain embodiments, an advantage of the above formulation is that the cost is separable. That is, with some additional mathematics, one can maximize subsequences independently. This property leads to an efficient dynamic programming algorithm. In certain embodiments, the algorithm is efficient (runs in low-order polynomial time and space) and guarantees an optimal solution.

Let c: [symbol] → [symbol] denote the maximum cost of a matching subproblem. In particular, let c(j,k,ƒ) denote the maximum cost of matching the peaks from 0 to k with the definition fragments from 0 to ƒ+1 in such a way that peak j matches size ƒ and peak k matches size ƒ+1. The cost of matching all sizes is therefore

$$c ( {M}^{*} ) = {max}_{j < k \leq {n}_{p}} c ( j , k , {n}_{s} - 2 ) \qquad ( 9 )$$

where M* is the optimal matching. Note that every definition fragment matches some peak, but only nₛ of the peaks need match in this embodiment.

One can now express a maximum cost recursively. For ƒ=0 there are no ratios to compute, so one need only be concerned with the height cost:

c(j,k,0)=(1−α)(cₕ(j)+cₕ(k)) (10)

For ƒ>0, one can compute the cost recursively by adding the height cost for the newly matched peak k to a new ratio cost and a previous subproblem cost:

$$c ( j , k , f ) = ( 1 - α ) \cdot {c}_{h} ( k ) + {max}_{i < j} \{ c ( i , j , f - 1 ) + α \cdot {c}_{r} ( i , j , k , f - 1 ) \} \qquad ( 11 )$$

Converting these equations to algorithms is straightforward. In certain embodiments, the size standard matching algorithm computes the individual elements in a consistent order. Furthermore, one may exploit the fact that one can match every size in the definition by limiting the computation. In certain embodiments, the size standard matching algorithm only needs to compute c(j,k,ƒ) for k>j>ƒ since j peaks cannot be made to fit all ƒ sizes if j<ƒ. Similarly, in certain embodiments, the size standard matching algorithm needs to examine only subproblems c(i,j,f−1) where i≧ƒ−1 since i peaks could not fit all ƒ−1 sizes if i<ƒ−1.

To this end, Algorithm 2 solves Equation 10 and Algorithm 3 solves Equation 11.

Algorithm 2 Basis of the Recursion (when ƒ=0)

for j←0,1, . . . , nₚ−2 do

for k←j+1,j+2, . . . , nₚ−1 do

c(j,k,0)←(1−α)(cₕ(j)+cₕ(k))

Algorithm 3 Compute Cost of Matching (when ƒ>0)

for ƒ←1,2, . . . ,nₛ−2 do 1.

for k←ƒ+1,ƒ+2, . . . ,ƒ+nₚ−nₛ+1 do 2.

for j←ƒ,ƒ+1, . . . , k−1 do 3.

c*←−∞

for i←ƒ−1,ƒ, . . . , j−1 do 5.

cᵢ←c(i,j,ƒ−1)+α·cᵣ(i,j,k,ƒ−1) 6.

if cᵢ>c* then 7.

c*←cᵢ 8.

c(j,k,ƒ)←(1−α)·cₕ(k)+c* 9.

As stated, these algorithms compute only the cost of an optimal matching. One will still retrieve a matching from this calculation. This is often a standard part of dynamic programming algorithms. When memory requirements are very high, it is often the practice to recompute the path to the optimal cost from the cost matrix. Since certain embodiments have relatively small sequences, one can trade time for memory by keeping an array of backpointers, or predecessors p. It is easy to maintain this array by adding the line p(j,k,ƒ)←i after line 8 in Algorithm 3. This assignment indicates that the predecessor to cost c(j,k,ƒ) is c(i,j,ƒ−1). Then, the size standard matching algorithm may reconstruct an optimal matching from Equation 9 by tracing backwards.

Computational Resources

### Theoretical Run-time Analysis

In certain embodiments, the algorithm's run time complexity is dominated by the number of times it executes Lines 6 and 7. The lines themselves execute in constant time. The inner (the i) loop executes them Σ_{t=ƒ-1}^{j-1} 1 times. Therefore the j loop executes them Σ_{j=ƒ}^{k-1} Σ_{i=ƒ-1}^{j-1} 1 times. The k loop terminates at k=ƒ+nₚ−nₛ+1=ƒ+m+1, where m=nₚ−nₛ is the number of extra peaks. Continuing in this way, one sees that the lines are executed a total of T(m, nₛ) times, where

$$T ( m , {n}_{s} ) = {\sum }_{f = 1}^{{n}_{s} - 2} {\sum }_{k = f + 1}^{m + f + 1} {\sum }_{j = f}^{k - 1} {\sum }_{i = f - 1}^{j - 1} 1. \qquad ( 12 )$$

This expression is not as formidable as it looks since the inner three summations are independent of the value ƒ. By a judicious substitution of variables, one sees that:

$${\sum }_{k = f + 1}^{m + f + 1} {\sum }_{j = f}^{k - 1} {\sum }_{i = f - 1}^{j - 1} 1 = {\sum }_{k = 0}^{m} {\sum }_{j = 0}^{k} {\sum }_{i = 0}^{j} 1. \qquad ( 13 )$$

A calculation shows that:

$$\begin{matrix}{\sum }_{k = 0}^{m} {\sum }_{j = 0}^{k} {\sum }_{i = 0}^{J} 1 = \frac{1}{6} ( {m}^{3} + 6 {m}^{2} + 11 m + 6 ) \\ = \frac{1}{6} ( m + 3 ) ( m + 2 ) ( m + 1 ) .\end{matrix}$$

It follows that

$$\begin{matrix}T ( m , {n}_{s} ) = {\sum }_{f = 1}^{{n}_{s} - 2} {\sum }_{k = f + 1}^{m + f + 1} {\sum }_{j = f}^{k - 1} {\sum }_{t = f - 1}^{j - 1} 1 \\ = \frac{1}{6} ( m + 3 ) ( m + 2 ) ( m + 1 ) ( {n}_{s} - 2 ) \\ = O ( {m}^{3} {n}_{s} ) .\end{matrix} \qquad ( 14 )$$

That is, the execution time increases only linearly with the number of definition fragments, but it increases as the cube of the number of extra peaks. Note that, when the number of peaks equals the number of definition fragments (that is, when m=0), Lines 6 and 7 are executed only nₛ−2 times, which is exactly the number of ratios that need to be compared to evaluate any matching.

### Empirical Measurements

The theoretical analysis in the previous subsection allows one to understand the asymptotic behavior of the algorithm. That is, it allows one to predict the trend in the run-time when the inputs are large. For smaller inputs, in certain embodiments, various overhead factors influence the run time.

One can construct several sets of synthetic data and time a C++ implementation of the algorithm. The data includes size standard definitions with nₛ=5 to nₛ=40 fragment sizes. In every case the ith fragment has size 20i,where i≧1. The in-lane peaks have positions equal to the definition sizes, but they also have m=0 to m=20 additional peaks, where the ith additional peak has position 10+20i, for i≧0. For each combination of nₛ and m, a test program executes the matcher component 20 times and divides the elapsed time by 20 also, to give the time for each execution in milliseconds.

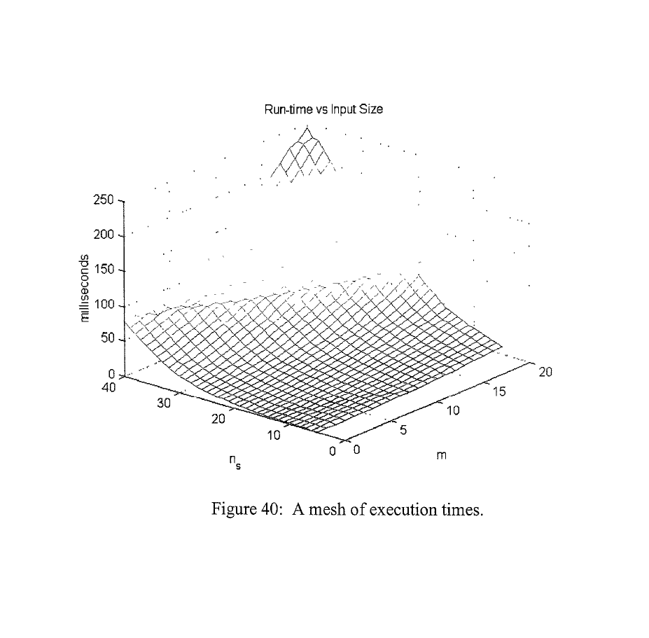

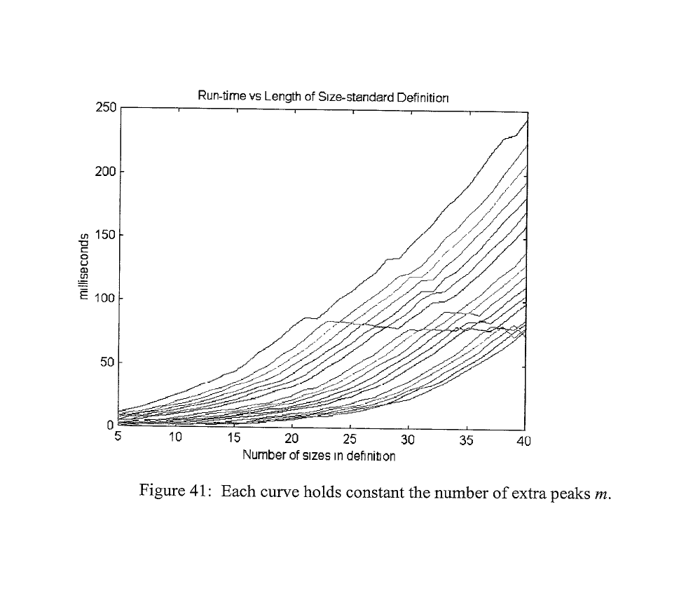

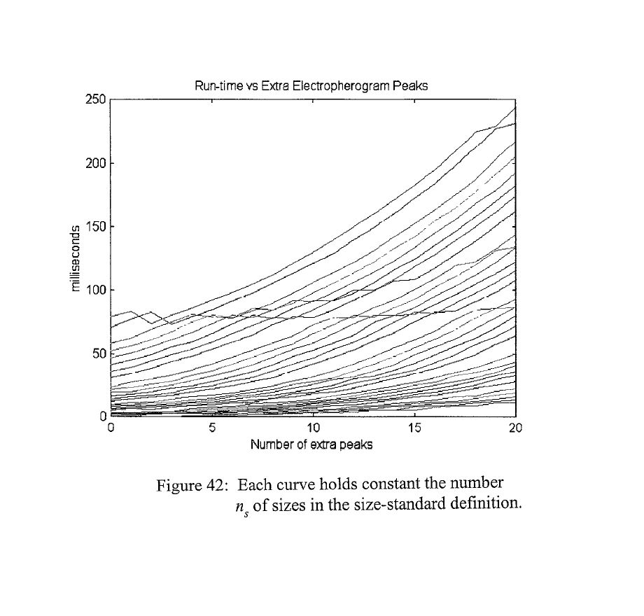

FIGS. 40 through 42 show the results. The execution times themselves, rounded to the nearest millisecond, are provided below.

### Memory

Full arrays for holding the costs and predecessors may use (m+nₛ)²nₛ=m²nₛ+2mnₛ²+nₛ³ real values. Initializing these arrays therefore takes asymptotically more time, when m³=O(nₛ²), than the optimization algorithm. If this is a problem, the arrays can be implemented as sparse arrays, so that they occupy O(m³nₛ) space as well as time. Another solution is to use full arrays, but to index them not with the peak indices, but rather with the substituted variables in Equation 13. A third possibility is to use and allocate full matrices, but to not initialize them.

Practical Considerations

In certain embodiments, one may want to determine a set of candidates peaks that the size standard matching algorithm should size. One may choose to allow a parameter m specifying the number of extra peaks to consider. In certain embodiments, the size standard matching algorithm then extracts the nₚ=nₛ+m tallest peaks from all peaks detected by the previous sizecalling step. In certain embodiments, one may use m=4. In certain embodiments, one may use a weighting factor a between ½ and ¾.

An analyst typically should choose a size standard definition that corresponds to the in-lane size standard. However, it may be that an analyst terminated a run early, before the longer fragments have had a chance to elute. In this case, the definition is not accurate, strictly speaking. To provide some robustness in this situation, one may test if the optimal matching satisfies a minimum acceptable quality parameter. If not, one may remove the last definition size and try again, repeating this process until the quality is acceptable. Alternatively, if the quality is unacceptable, one may simply report this without returning a matching.

SIZE CALLING

In certain embodiments, the system uses a size calling algorithm. The size calling algorithm predicts the nucleotide size corresponding to data peaks from a sample in view of the standard sizes.

In certain embodiments, such an algorithm uses at least one of five size calling algorithms: local southern, global southern, second order least square, third order least square, and cubic spline interpolation.

In certain embodiments, the size-calling algorithm maps scan numbers (read-frames, data points, etc.) into fragment sizes. In certain embodiments, the size calling algorithm provides global (or least squares fit) methods and local (or interpolation) methods. In certain embodiments, the size calling algorithm includes three global methods (second order least squares, third order least squares, and global Southern) and two local methods (cubic spline and local Southern).

Global Methods

In certain embodiments, the global methods determine the size ƒ(x) of a fragment at scan number J: by evaluating a function ƒ. The function depends on the method:

second order polynomial:

ƒ₂(x)=ax²+bx+c

third order polynomial:

ƒ₃(x)=ax³+bx²+cx+d

global Southern:

ƒ₂(x)=k₁/(m−m₀)+k₂,

where mobility m=1/x.

Before a function can be evaluated, it typically is fit to the data. In certain embodiments, the goal of each global fitting method is to find coefficients (a, b, m₀, . . . ) that minimize the sum of errors squared. That is, given a set of matched size-standard pairs {(xᵢ, yᵢ): i=1,2, . . . , n}, (x=standard scan numbers, y=standard sizes), find coefficients for f that minimize the sum:

$${\sum }_{i = 1}^{n} {e}_{i}^{2} ,$$

where eᵢ=yᵢ−ƒ(xᵢ). Standard methods may be used to accomplish this task. See, e.g., W. H. Press, B. P. Flannery, S. A. Teukolsky, and W. T. Vetterling, General linear least squares, In Numerical Recipes in C, chapter 14.3, pages 528-539, Cambridge University Press, 1988.

Local Methods

Cubic Spline

A cubic spline is meant to simulate numerically a draftsman's mechanical spline. In certain embodiments, it connects every adjacent pair of dots with their own cubic polynomial. In certain embodiments, it ensures that two curves that share a dot have the same value, first derivative, and second derivative at that dot. In certain embodiments, these constraints nearly determine the solution. In certain embodiments, the final constraint is that the size calling algorithm uses a so-called natural spline, for which the second derivative at the endpoints is 0. In certain embodiments, the size calling algorithm represents these constraints as a set of linear equations, which it then solves with Gaussian elimination (W. H. Press, B. P. Flannery, S. A. Teukolsky, and W. T. Vetterling, General linear least squares, In Numerical Recipes in C, chapter 14.3, pages 528-539, Cambridge University Press, 1988.).

Local Southern

For autoradiograms, mobility m is proportional to the distance of the migrated isotope from the injection well (since time is fixed). Southern (Southern, Measurement of DNA length by gel electrophoresis, Analytical Biochemistry, 100:319-323 (1919)) noticed that the fragment size versus 1/m is (almost) a straight line:

ƒₛ(m)=k₁/m+k₂.

Only the high mobility (short) fragments did not fit this linear prediction. To account for these high mobility fragments, Southern introduced an initial mobility m₀ into the equation:

ƒₛ(m)=k₁/m−m₀)+k₂ (5.1)

In certain embodiments, a scan number x corresponds to time (since the capillary length, or well-to-read distance, is fixed), and so is inversely proportional to mobility. For simplicity, one may set m=1/x.

Given a scan number x, in certain embodiments, the size calling algorithm (the local Southern method) finds size-standard fragments a, b, c, and d so that scan x is between scans b and c. These fragments have known sizes ƒₛ(1/a), ƒₛ(1/c), ƒₛ(1/d), and ƒₛ(1/d) respectively. In certain embodiments, the size calling algorithm then sets up a system of three equations, with m=1/a, m=1/b, and m=1/c in Equation 5.1, and solves them exactly for k₁, k₂, and m₀. Once it has these values, in certain embodiments, it interpolates the curve at m by evaluating the resulting equation ƒₛ₁(m) at m=1/x.

In addition, in certain embodiments, the size calling algorithm sets up another system of three equations, with m=1/b, m=1/c, and m=1/d in Equation 5.1, and solves these exactly for k₁, k₂, and m₀. It then evaluates Southern's equation ƒₛ₂(m) at m=1/x. Finally, in certain embodiments, the size calling algorithm averages the two resulting sizes, so that the fragment size with mobility m is (ƒₛ₂(m)+ƒₛ₂(m))/2.

The Solution at the Limit

There is a potential problem not addressed in Southern's paper (Southern, Measurement of DNA length by gel electrophoresis, Analytical Biochemistry, 100:319-323 (1919)). To see it, rewrite Southern's Equation 5.1 by renaming ƒₛ₂(m) as y, k₁, as k, and k₂ as y₀.

y=k/(m−m₀)+(y₀.

An easy rearrangement gives the equation:

(y−y₀)(m−m₀)=k₁.

which makes it clear that Southern's equation describes a hyperbola. Now, a hyperbola describes a straight-line segment only at the limit. More to the point, suppose (m₁, y₂), (m₂, y₂), and (m₃, y₂) are three collinear points. There are no finite constants k, mc, and yc such that Equation 5.2 goes through all three points (m₁, y₁), (m₂, y₂), and (m₃, y₃). Such a situation might and does arise in fragment analysis applications, and so is addressed.

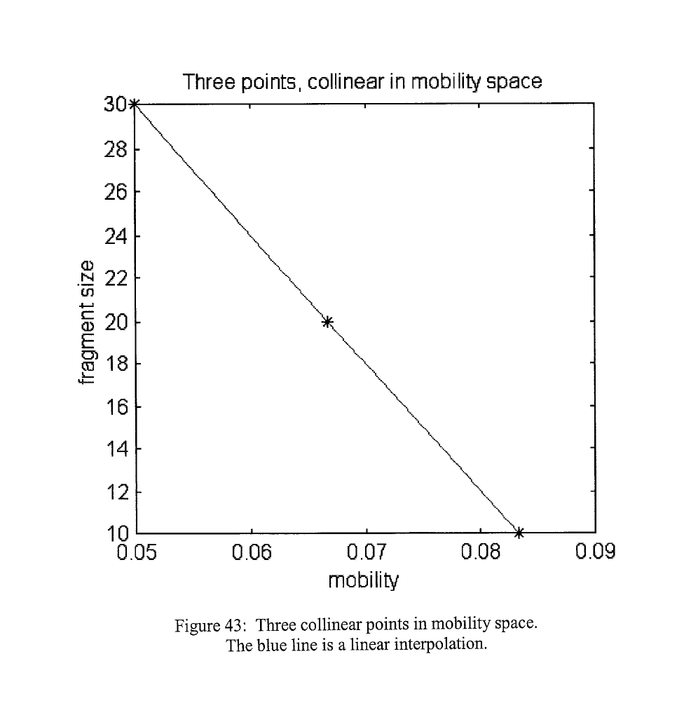

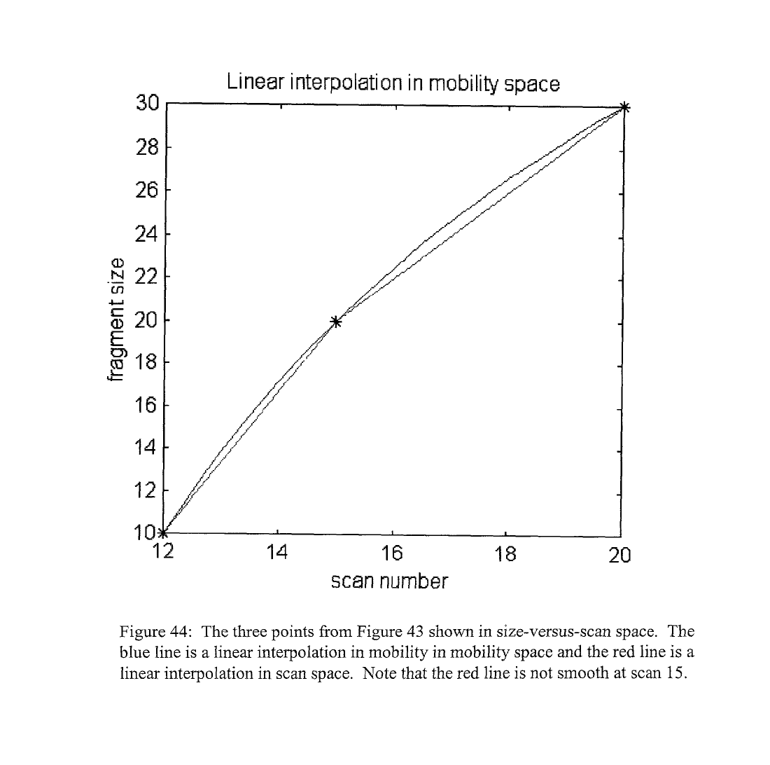

In certain embodiments, the size calling algorithm detects such collinear triplets and interpolates linearly in size-versus-mobility space (mobility space) to call a size. For example, suppose one has size standard fragments at 10, 20, and 30 base pairs, and that they elute at 12, 15, and 20 scans respectively. They then have mobilities of 1/12, 1/15, and 1/20 capillary lengths per scan. These points are collinear in mobility space, as shown in FIG. 43.

Note that collinear points in mobility space are not collinear in scan-versus-mobility space (scan space), as shown by the example in FIG. 44. Therefore, it would be incorrect for the size calling algorithm to treat such points by interpolating linearly in scan space.

On the other hand, suppose the size calling algorithm encounters three points that are collinear in scan space. Such points are not collinear in mobility space, and Southern's equation (Equation 5.1) applies without change. Southern's equation would interpolate such points linearly in scan space, resulting in a smooth curve (a line segment, in fact) as expected.

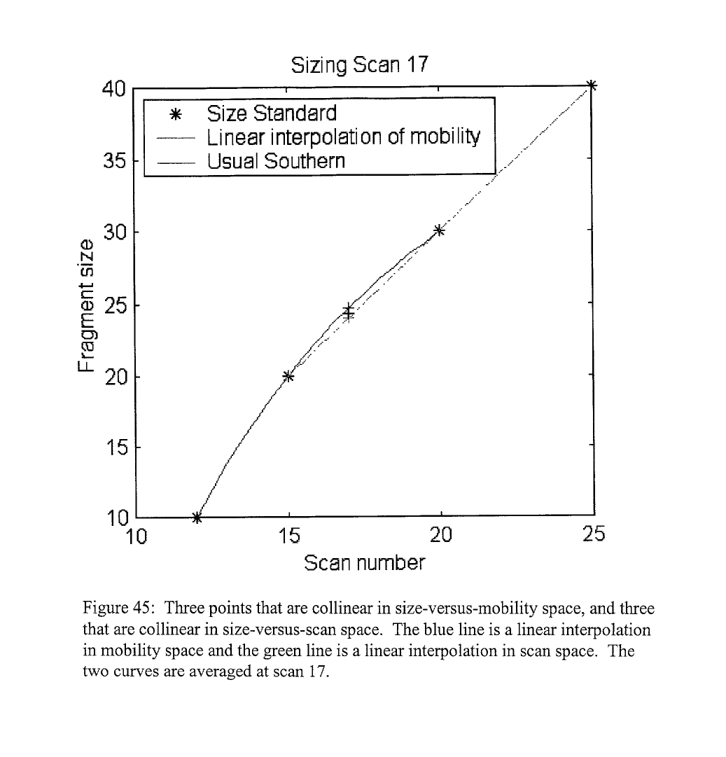

FIG. 45 shows both cases, and shows how the size calling algorithm in certain embodiments would size a fragment at scan 17. The leftmost three size-standard points are collinear in mobility space, while the rightmost three points are collinear in scan space. In certain embodiments, the size calling algorithm obtains the blue ‘+’ at scan 17 by linear interpolation in mobility space. In certain embodiments, it obtains the green ‘+’ by solving a system of three Southern equations. It then sizes the fragment at scan 17 by averaging these two sizes, as shown by the black ‘+’.

ALLELE CALLING

In certain embodiments, the system uses an allele calling component. Such a component is used to interpret what data actually corresponds to alleles. In certain embodiments, one uses one or more algorithms to determine the data points that actually correspond to an allele.

In certain embodiments, one uses more than one allele calling algorithm and the component uses that combined information in a committee approach to provide the allele call. In certain embodiments, one may use a single allele calling algorithm.

The following description of certain embodiments involves allele calling when one analyzes dinucleotide repeats at given loci using PCR amplification. The invention is in no way limited to such work and may involve any number of repeats or may involve other types of genetic polymorphisms. Other polymorphisms include, but are not limited to, SNP's (single nucleotide polymorphisms), single base insertions and deletions, insertions and deletions involving more than one base, and rearrangements.

Similarly, embodiments of the algorithms may be applied to other types of data in which multiple algorithms produce results that typically require interpretation and scoring in terms of their confidence values. Such other areas of application include, but are not limited to, the following: basecalling (de novo, mixed base and comparative sequence); SNP basecalling; spot-finding for microarrays; protein sequencing; protein/gene expression; peptide searches (a noisy time series alignment problem); and modeling of biological systems. One skilled in the art will appreciate all of the many types of nucleic acid and amino acid information that may be evaluated according to the present inventions. Examples include, but are not limited to, data from any of the applications above and any evaluation of properties including nucleic acid or amino acid length, molecular weight, or nucleic acid or amino acid identity.

In the committee approach for all of these applications of interpreting data, one uses the output of more than one algorithm rather than relying upon but one algorithm. Often, different algorithms may have various advantages over others depending on various conditions. The committee approach uses different algorithms to generate a meaningful confidence value on the correct interpretation of multiple data points. According to certain embodiments, the committee approach is particularly powerful when combined with the concept of establishing the operating environment first, an example of which is illustrated by the Envelope Caller described herein.

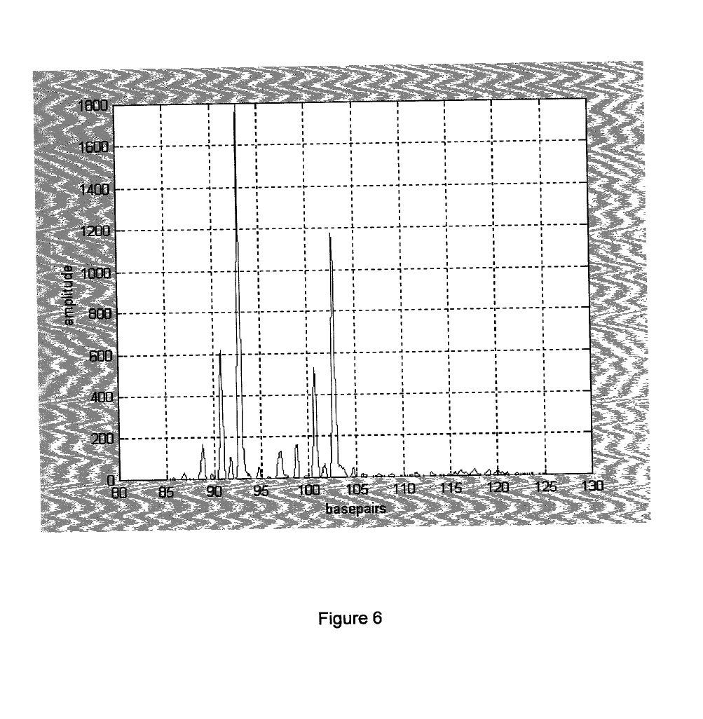

To determine given alleles at various loci, one can use PCR to selectively amplify regions of the gene that are known to have different alleles. In this example, one attempts to locate different length dinucleotide repeats at given loci. U.S. Pat. No. 5,580,728 describes certain methods that can be used according to the present invention to amplify the genetic material in a sample and to obtain data that correlates to the different lengths of amplified nucleic acids. U.S. Pat. No. 5,580,728 and all documents cited therein are expressly incorporated by reference herein. Possible data that may be generated is shown in FIG. 6.

FIG. 6 illustrates results that include artifacts created by the PCR amplification process. Without such artifacts, that data would show peaks at 93 and 103 basepairs, which would indicate that the individual is heterozygous for the two alleles of size 93 and 103 basepairs. PCR stutter, however, introduces additional peaks at 91 and 89 for the allele at 93, and at 101, 99, and 97 for the allele at 103. The stutter results in fragments that are shorter by one or more dinucleotides than the actual allele in the sample. Also, during the PCR process, additional A nucleotides may be added, which results in artifacts in FIG. 6 having an extra basepair (i.e., at 94 for the allele at 93 and at 104 for the allele at 103). FIG. 6 shows a relatively simple pattern that represents a heterozygous individual with alleles 93 and 103 and that includes artifacts. The artifacts that may be introduced, however, are not always simply disregarded when the actual alleles are closer together and allele signatures overlap. Thus, the present invention provides systems for interpreting data and making correct allele calls.

PCR stutter and the addition of A nucleotides is discussed in U.S. Pat. No. 5,580,728. That patent discusses a particular algorithm that may be used to try to make correct allele calls. The present invention provides typically more reliable allele calling. The present invention includes not only new algorithms, but also systems that use more than one algorithm to increase the reliability of the call.

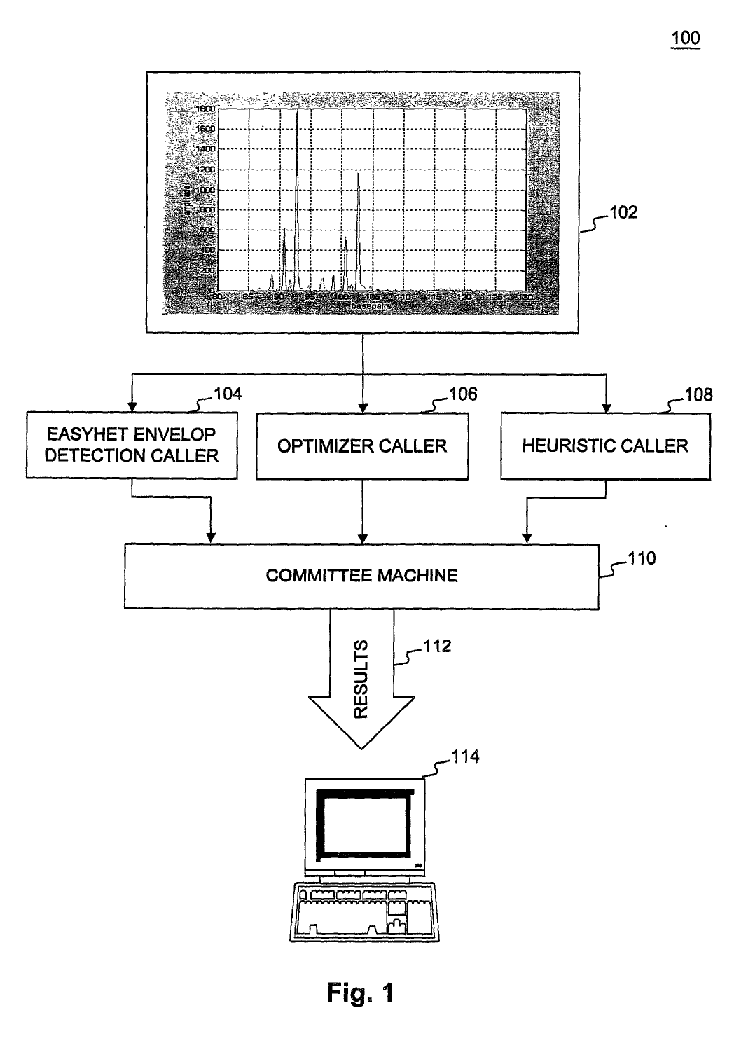

FIG. 1 depicts an overview block diagram of a committee system 100 in which methods and systems consistent with the present invention may be implemented. Data 102 includes typical size-called data from a DNA sequencer such as the ABI 3700 DNA sequencer (Applied Biosystems). Data 102 may be passed to multiple allele calling algorithms, such as the Envelope Detection Caller algorithm 104, Optimizer Caller algorithm 106, and a Heuristic Caller algorithm 108. Envelope Detection Caller algorithm 104 detects a heterozygous allele pattern when alleles are well separated spatially. Optimizer Caller algorithm 106 identifies impulse functions (e.g., location of the allele peaks) given a response signal (e.g., a raw microsatellite signal). Heuristic Caller algorithm 108 uses multiple rules and filters to eliminate peaks that are not alleles from consideration. More information on algorithms 104, 106, and 108 is provided below.

Each algorithm reports their results to a committee machine 110 that uses logic and/or rules to assign a confidence level to the call. Committee machine 110 produces robust results and can predict calls. That is, machine 110 receives call results from several callers and can provide a degree of confidence for the resulting calls based on a statistical probability of an answer being correct given the degree of consensus between the different callers. More information on the committee of experts is further described below. The confidence level may be created by considering agreement between calling algorithms 104, 106, and 108. Results 112 contain the confidence level for each test performed by committee machine 110, and results 112 are transmitted to a user of a computer 114.

The committee system 100 provides a number of benefits over traditional allele calling algorithms. First, since each algorithm uses a different strategy in determining whether there is a call, if all the callers agree, then an extremely high value of confidence may be placed on the calls. If, however, not all allele calling algorithms agree, differing levels of confidence may be placed on the calls depending upon which algorithms agree. By considering the level of agreement between the different algorithms over a large population of data, statistically significant confidence values can be assigned to the allele calls.

I. Committee Allele Calling System Operation

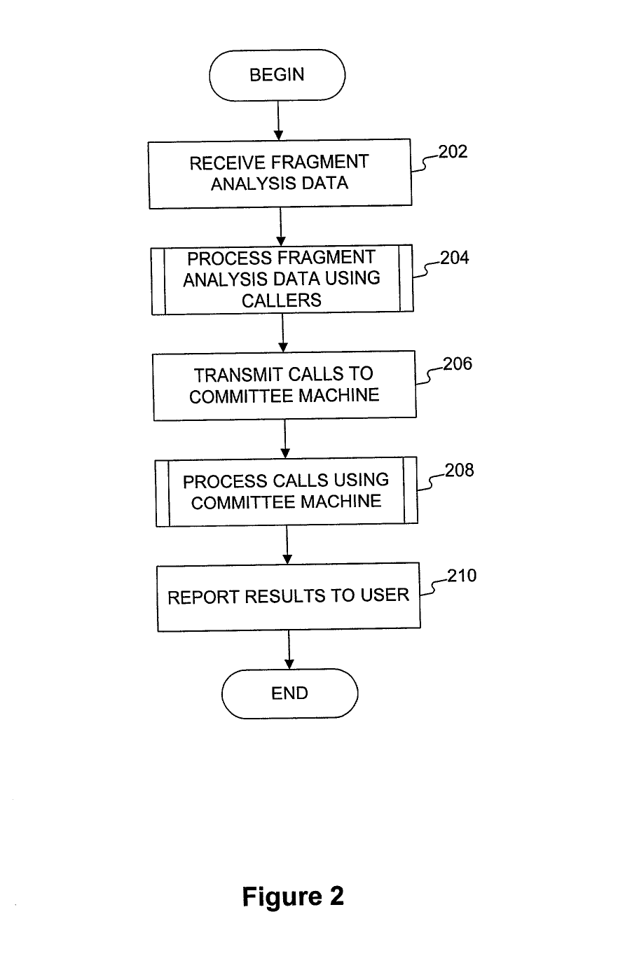

FIG. 2 depicts a flow chart of the steps performed by a data processing system in processing allele calls according to certain embodiments. First, the data processing system receives size-called fragment analysis data (step 202).

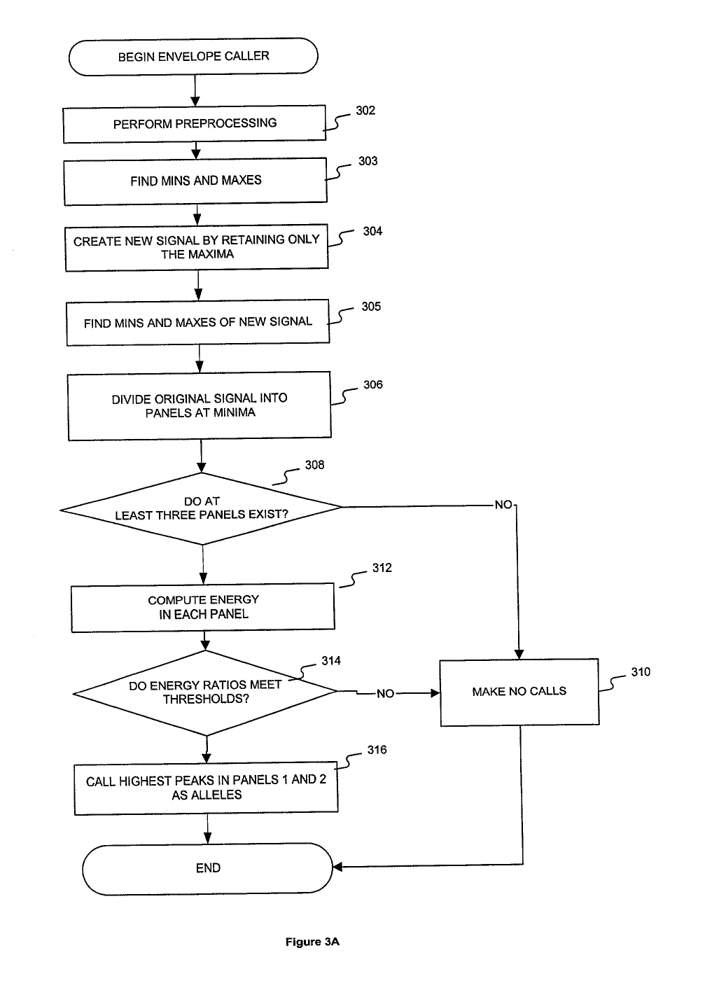

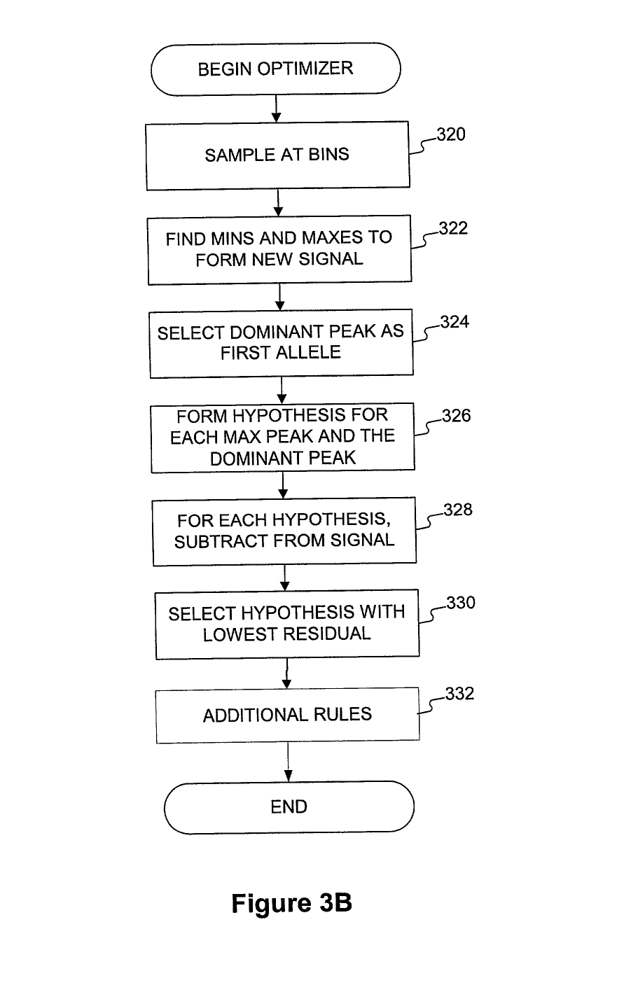

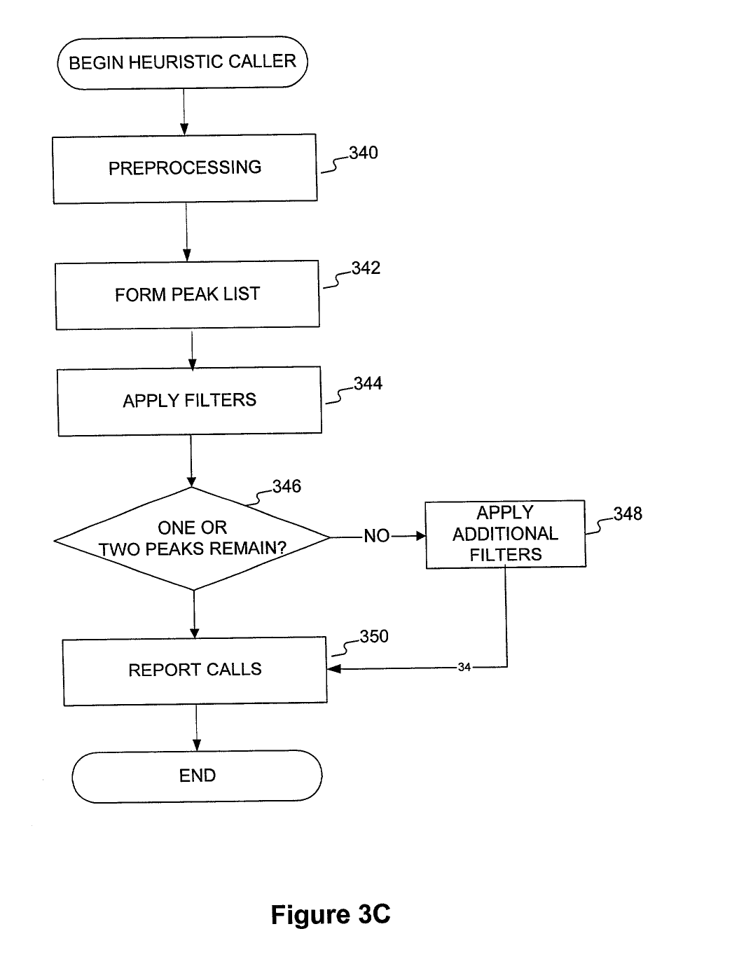

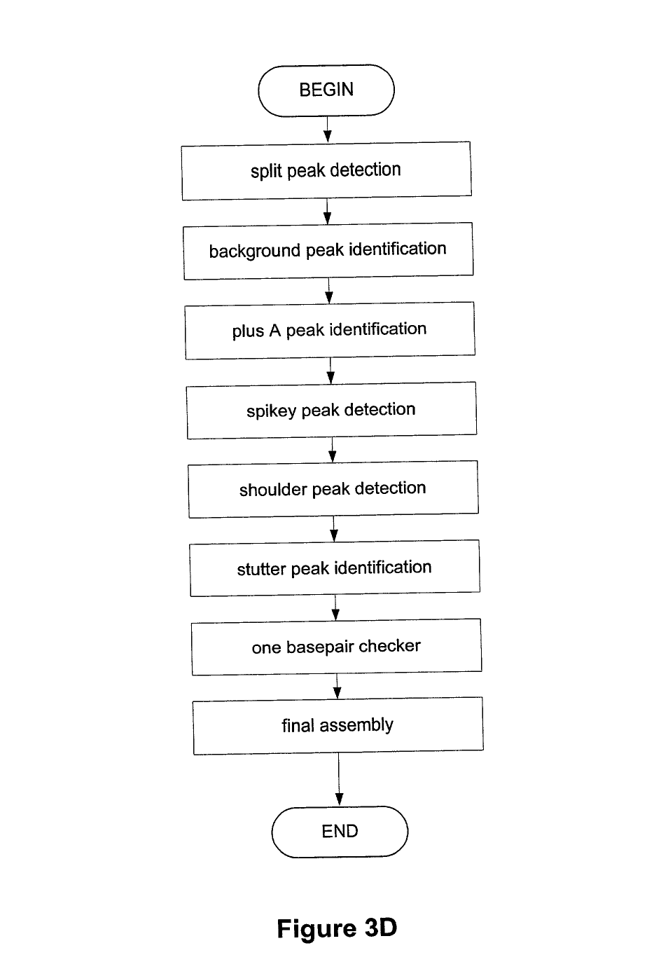

The received data may then be processed using various allele calling algorithms (step 204). Each caller algorithm operates well in different environments and on different signals. By using more than one caller on the same set of data, committee machine 110 may assign a confidence level to the call. Algorithms may either examine the data's complexity, and should it pass certain requirements, make the appropriate calls or make the calls regardless of data complexity. Several exemplary calling algorithms are described in FIGS. 3A-3D.

Once the data is analyzed with each allele calling algorithm, the results of each call are transferred to a committee machine 110 (step 206). Committee machine 110 processes the results of the calls (step 208) and arbitrates a decision and assigns an appropriate confidence value for the results of the calling algorithms. The results of this arbitration are reported to a user as the fragment lengths (calls) accompanied by a confidence value (step 210).

II. Envelope Caller

FIG. 3A depicts a flow chart of the steps performed by a data processing system when processing alleles with the Envelope Caller algorithm according to certain embodiments. The Envelope Caller algorithm typically is used to detect a heterozygous allele pattern where the alleles are well separated spatially. The Envelope Caller assesses the complexity of a signal from the nucleic acid sequencer prior to making a call and if the signal's complexity is below a threshold (i.e., the signal is in the caller's operating region) it makes the call. Thus, since the caller operates in a constrained region where it knows it stands an excellent chance of being correct, the call is may be extremely accurate.

First, the algorithm may perform preprocessing such as smoothing (step 302). For example, the algorithm may use N-point smoothing replacing each point with a local average over itself and N points on either side. By replacing each point with such a mean, noise is removed from the signal, and a smoother signal remains.

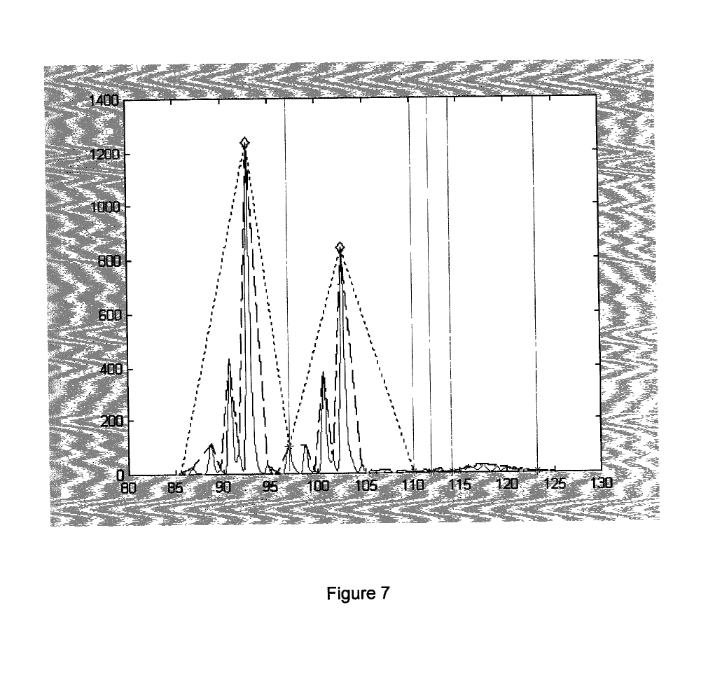

Next the minima and maxima of the signal are determined (step 303) using a technique such as the Savitzky-Golay algorithm (See, e.g., Numerical Recipes in C: The Art of Scientific Computing, William H. Press, Saul A. Teukolsky, WIlliam T. Vetterling, Brian P. Flannery, Cambridge University Press, 1992, pages 650-655) which uses calculation of the derivatives of the signal in its processing. Other peak detection methods may be used. This step reduces the signal's dimensionality significantly by effectively expressing the signal's general shape with fewer points. The effect of this can be seen in FIG. 7. Here the original signal is the solid line. After calculation of the minima and maxima, the signal is represented as the broken line.

In step 304, a new signal is formed by retaining only the maxima. This has the effect of determining the envelope of the signal. In FIG. 7, this signal is shown as the dotted line. Next, the signal is passed back through the algorithm that determines the minima and maxima (step 305). With this new representation the original signal is then divided into panels at each minimum (step 306). A panel is a large section of the signal that is bounded by the signal's deep local minima. In FIG. 7, 6 panels exist and are bounded as outlined in table 1.

TABLE 1

| Panel | Boundaries (basepairs) |
|---|---|
| 1 | 80-97 |
| 2 | 97-110 |
| 3 | 110-112 |
| 4 | 112-115 |
| 5 | 115-123 |
| 6 | 123-130 |

In order to determine the signal complexity and whether or not the algorithm should make a call, the algorithm first determines if three panels exist (step 308). If, at least three panels exist, the algorithm computes an energy level for each panel, for example, by summing the square of each element in the panel (step 312). Other methods of assessing the signal's energy in defined regions may be used. Since the algorithm is searching for the envelope characteristic of two well separated alleles, one typically uses three panels to ascertain if two distinct allele signatures exist. When one is searching for X number of alleles, one typically uses X+1 panels to ascertain if X distinct allele signatures exist.

Using the three largest energy levels (E1, E2, and E3, respectively—which in the figure correspond to panels 1, 2, and 5), the Envelope Caller algorithm performs a “threshold” determination (step 314). That is, using the three energy levels (E1, E2, and E3), the algorithm determines, for example in certain embodiments, whether E2 is greater than 20% of E1, and whether E3 is no more than 7% of E2. If these conditions exist in these embodiments, the signal is of sufficiently low complexity that the envelope caller can operate. The calls are then made by reporting the largest peaks in each of the panels with the greatest energy. Thus for the case illustrated in FIG. 7, the calls would be made at the peaks topped by the diamond symbol at 93 and 103 basepairs.

In summary, certain embodiments of the Envelope Caller may include the following:

1. Pass the signal through a min/max detection algorithm and discard the minima. Thus, an envelope of the signal is obtained by connecting the points that are maximal.

2. Pass this new signal through a min/max detection algorithm again.

3. Divide the signal into panels of interest using the min/max information. A panel of interest here is defined as one where the signal is initially low, then increases rapidly, and falls off again towards the baseline. In these embodiments, the energy in these regions is calculated by summing the squares of the data in these regions.

4. Consider only the three regions with the greatest energy.

5. Choose the two dominant peaks in the signal and that the signal represents a heterozygous condition. In such a case, the allele calls are the maxima in the two panels with the greatest energy.

The following code may be used according to certain embodiments of the Envelope Caller methods.

Line 6 calls the subroutine envelope (lines 21-53) which divides the signal into the panels and calculates the energy of the panels and then identifies the three panels with the greatest energy content. Line 10 tests the condition given in step 4. If these conditions are met, line 11 retrieves the allele calls.

| | |
|---|---|
| 1 | d = fieldnames(D); |
| 2 | ind = [ ]; |
| 3 | |
| 4 | for i=1:size(d,1), |

| | |
|---|---|
| 5 | eval([‘cur=D.’ char(d(i)) ‘;’]); |
| 6 | [A, h, p1, p2]=envelope(cur); |
| 7 | |

| | |
|---|---|
| 8 | if size(A,1)>3, |

| | |
|---|---|
| 9 | B(i,1:2)=[A(2,5)/A(1,5) A(3,5)/A(2,5)]; |
| 10 | if (A(2,5)/A(1,5) > 0.2) & (A(3,5)/A(2,5) < 0.07), |
| 11 | [peak_ind height]=getEnvelopeCalls(A,cur.analyzed); |
| 12 | R(i).alleleList=[cur.analyzed(peak_ind,1) height’]; |
| 13 | else |
| 14 | R(i).alleleList=[ ]; |
| 15 | end |

| | |
|---|---|
| 16 | end |
| 17 end | |
| 18 | |
| 19 | |
| 20 | %%%%%%%%%%%%%%%%%%%%%%%%%%%%%%%%%%%%%%%%%%%%% |
| 21 | function [A, h1, p1, p2] = envelope(cur, plotflag) |
| 22 | |
| 23 | % function h1=divider(cur, plotflag) |
| 24 | % |
| 25 | % cur - structure containing the data |
| 26 | % plotflag - set to anything to plot the process |
| 27 | % |
| 28 | % h1 - vector of division points |
| 29 | % |
| 30 | % |
| 31 | |
| 32 | anal = cur.analyzed; |
| 33 | f = peak_trough(anal(:,2)); % first pass through the min/max detector |
| 34 | p1 = [f.maxvals]; p2=[f.maxinds]; |
| 35 | g = peak_trough(p1); % second pass through min/max detector |
| 36 | h1 = anal(round(p2(round(g.mininds))),1); |
| 37 | ind = [1 closest(cur.analyzed(:,1), h1) length(anal)]; |
| 38 | |
| 39 | for i=1:length(ind)−1, |
| 40 | |
| 41 | A(i,1:2) = ind(i:i+1); |
| 42 | A(i,3) = diff( ind(i:i+1) ); |
| 43 | A(i,4) = diff( [anal(A(i,1),1) anal(A(i,2),1) ]); |
| 44 | A(i,5) = sum(anal(A(i,1):A(i,2), 2).\hat{ }2); |
| 45 | A(i,6) = A(i,5)/A(i,4); |
| 46 | |
| 47 | end |
| 48 | |
| 49 | [p ind]=sort(A(:,5)); |
| 50 | A=A(flipud(ind),:); |
| 51 | |
| 52 | if exist(‘plotflag’), plotDivisionLines(cur,A,h1,p1,p2); end |
| 53 | |
| 54 | %%%%%%%%%%%%%%%%%%%%%%%%%%%%%%%%%%%%%%%%%%%%% |
| 55 | function [peak_ind, height] = getEnvelopeCalls(A, cura) |
| 56 | |
| 57 | for i=1:2, |

| | |
|---|---|
| 58 | [height(i) peak_ind(i)]=max(cura(A(i,1):A(i,2),2)); |
| 59 | peak_ind(i)=peak_ind(i)+A(i,1); |

| | |
|---|---|
| 60 | end |

III. Optimizer Caller

U.S. Pat. No. 5,580,728, which is incorporated by reference, describes allele calling via deconvolution. This is similar to the Optimizer Caller algorithm consistent with certain embodiments of the present invention.

According to certain embodiments the Optimizer Caller operates as follows. The algorithm operates on the principle of deconvolution that identifies the impulse functions (location of the allele peaks) given the response signal (the raw microsatellite signal). The routine uses model fit optimization to effect deconvolution. The model parameters optimized are peak location, peak height, and stutter percentage.

According to certain embodiments, the algorithm first performs dimensionality reduction by sampling at bins and then identifies the largest peak as the dominant allele. Bins are locations where one would expect to find alleles. Due to the way the data is generated, fragment lengths seldom are reported as integer base pairs. Thus, any peak falling within some threshold of the center of the bin is said to be that length. In certain embodiments, this threshold is +/−0.15 basepairs. Thus, if a peak were to be size-called at 100.87 basepairs and a bin existed at 101 bp, the peak would be reported as 101 bp.

Sampling at the bins allows one to eliminate data points from the analysis. Bins are determined by previously compiled data. For example, one may pass to the system an original set of bins based on previously compiled statistics that reflect expected allele locations, and a sampling grid is formed by interpolating a one basepair grid that accomodates these bins. This creates a continuum of bins spaced at one basepair intervals upon which the signal is sampled.

Through building models where the amount of stutter is varied, the algorithm selects the next most likely allele by choosing the impulse function whose model results in the lowest residual error when subtracted from the original signal.

The flowchart in FIG. 3(B) according to certain embodiments illustrates the concept as follows:

1) Sample at the bins (320)—as discussed above, the bins are locations where one would expect to find alleles. Thus, the signal above is sampled at these locations. Typically these locations includes minima and maxima but will also contain other portions of the signal (flat regions, stutter peaks).

2) Find minimas and maximas (322)—using the Savitsky-Golay approach, the precise location of the minima and maxima are located. The maxima represent possible alleles.

3) Select dominant peak as one allele (324)—typically, the largest peak is an allele—selecting this peak is a safe strategy, the problem is now reduced to finding the other allele (it if is present).

4) Form a series of hypotheses (models) by varying the location of the secondary peak and the amount of stutter in both the dominant and secondary peaks (326).

5) Subtract each model from the signal found in step (2) (328). The residual is kept in a table.

6) Select model with the lowest residual (330)—the model that results in the lowest residual best describes the signal from step (2) and thus is declared the winner. The allele calls are the location of the alleles that resulted in the model.

7) Transmit calls to user after application of any additional rules (332) such as removing left peaks below a certain threshold—experimentation has shown that peaks below a certain threshold are usually noise.

According to certain embodiments, the main Optimizer Caller algorithm steps are summarized as follows:

1) Data Reduction:

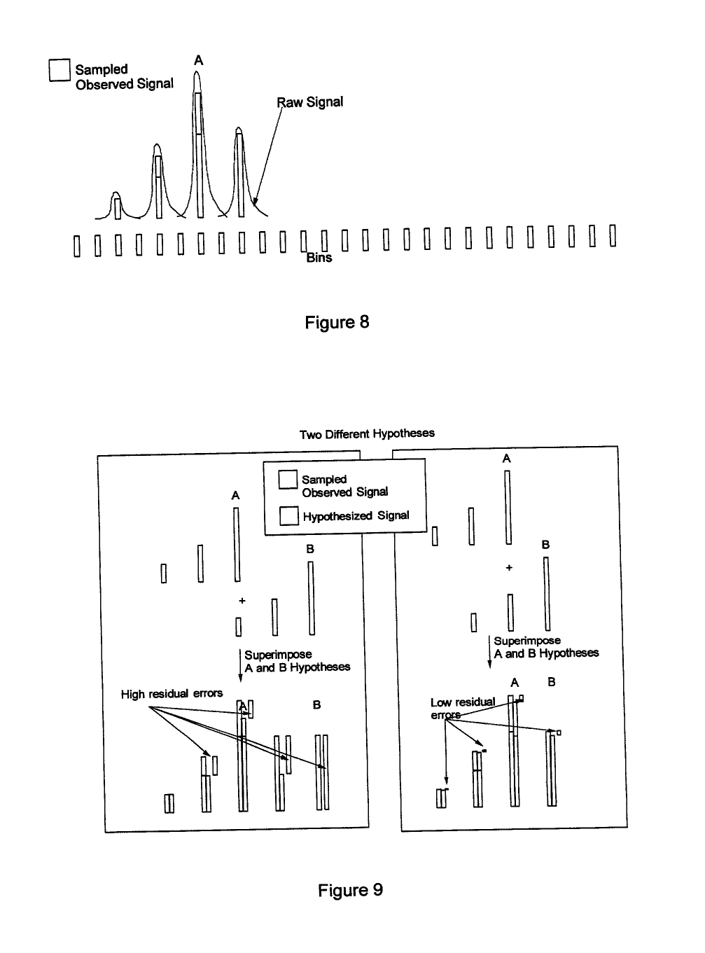

Using the a priori bins passed in, a sampling grid which includes additional bins is constructed. Then the signal is sampled to give a simplified discrete representation of the microsatellite signal, essentially the peak heights at the centers of the bins. See FIG. 8.

2) Find the highest peak and assume it is one of the allele peaks, the “A” allele. See FIG. 8.

3) Search for the B allele:

The algorithm searches for the location, height, and stutter percentage of the B allele peak that minimizes the residual signal, that is, the signal left over after subtracting the hypothesized signal from the observed signal. (The B peak may in fact be the same as the A peak, i.e. a homozygote.)

FIG. 9 illustrates two different attempts in the search for the B allele. Recall that the A allele has been assumed to be the highest peak. Different hypotheses for the location, height, and stutter percent for the B allele peak are made. A composite signal is generated by superimposing the A and B hypotheses. The hypothesized signal is then compared to the observed signal and a residual error is calculated. The hypothesis with the lowest residual error is reported as the B allele.

The method used to search for the best B allele parameters is flexible. In the first implementation of this algorithm, simple heuristics were used to prune the search space, but it was essentially an exhaustive search for the best B allele. Methods such as conjugate gradient, simplex or simulated annealing could be applied.

IV. Heuristic Caller

FIG. 3C depicts a flow chart of the steps performed by a data processing system when processing alleles with the Heuristic Caller algorithm according to certain embodiments. The Heuristic Caller algorithm uses multiple rules (filters) to eliminate peaks that are not alleles. By removing the peaks using the filters, the remaining peak(s) may be alleles.

First, any of a number of preprocessing steps may be performed. Examples include the N-point smoothing mentioned in the Envelope Caller or noise quantification (or Noise Checker). Noise quantification is used to assess the quality of the signal. An example of Noise Quantification includes:

1) taking the signal;

2) performing smoothing as in 302 of FIG. 3A;

3) subtracting the smoothed signal from the original signal; and

4) summing the squares of the difference between the two signals to get the sum squared error (SSE).

If the signal is relatively noise free, the SSE will be low and more faith can be placed in the calls. If the SSE is high then the user is alerted that it might be wise to look at the signal and make calls manually.

After any such preprocessing steps according to certain embodiments, the process includes step 342 where the Heuristic Caller algorithm forms a peak list using a peak detection algorithm such as the Savitzky-Golay algorithm. According to certain embodiments, a list is formed with an entry for each peak that contains the following three pieces of information; peak location, peak height, and peak width. Next, various filters are applied to remove peaks that are not the correct allele calls (step 344).

Nonlimiting examples of one or more rules that may be employed include:

Remove split peaks (Split peak checker)

Remove background peaks (Background peak checker)

Remove peaks due to plus A distortion (Plus A Checker)

Remove spikey peaks (Spike peak checker)

Remove shoulder peaks (Shoulder peak checker)

Remove stutter peaks (Stutter checker)

Split peaks are two peaks that appear in the peak list that are similar in height (for example, at least about 70%) and typically less than about 0.1 basepairs apart. They typically are caused by a mixture of double and single stranded DNA. According to certain embodiments, if split peaks are detected, only the highest of the split peaks is preserved.

Background peaks are spurious peaks that do not have any significant stutter. Stutter almost always occurs for dinucleotide markers. Thus, peaks that do not have any significant stutter are considered background peaks and are removed from the list. Background peaks are typically due to sample contamination.

Spikey peaks are spurious peaks that are tall but have a width that is not typical of the other peaks. A peak list has height, width and location data. Thus, an average peak width can be determined and any peaks that are too narrow compared to the rest of the population are removed. They are typically caused by sample contamination.

Shoulder peaks are peaks that appear very close to another peak and thus have the appearance of a shoulder. They are similar to spikey peaks except typically are lower in height, greater than 0.1 bp away, and less than 1 bp away. These are often caused by instrument noise. In certain embodiments, the shoulder peaks are removed.

According to certain embodiments, the filters applied in step 344 include at least one of those shown in the flow chart of FIG. 3D. The One basepair Checker checks neighboring peaks to see whether there are one basepair peaks present. In certain embodiments, one may change the order of the filters. For example, according to certain embodiments, the Plus A checker and the Shoulder peak checker are switched with one another in the flowchart of FIG. 3D. (The Final Assembler shown in FIG. 3D assembles the final results and calls the alleles.)

Once all non-allele peaks are removed the Heuristic Caller algorithm determines if there are one or two remaining peaks (step 346). If there are more than two remaining peaks, additional filters are applied (step 348) in order to reduce the number of peaks to one or two. These rules are based on special cases that have been determined via observation. A nonlimiting example of a rule would be when four peaks remain—generally, the lowest two can be removed. Once only one or two peaks remain, they are designated as the allele calls and are passed to the committee machine (step 350).

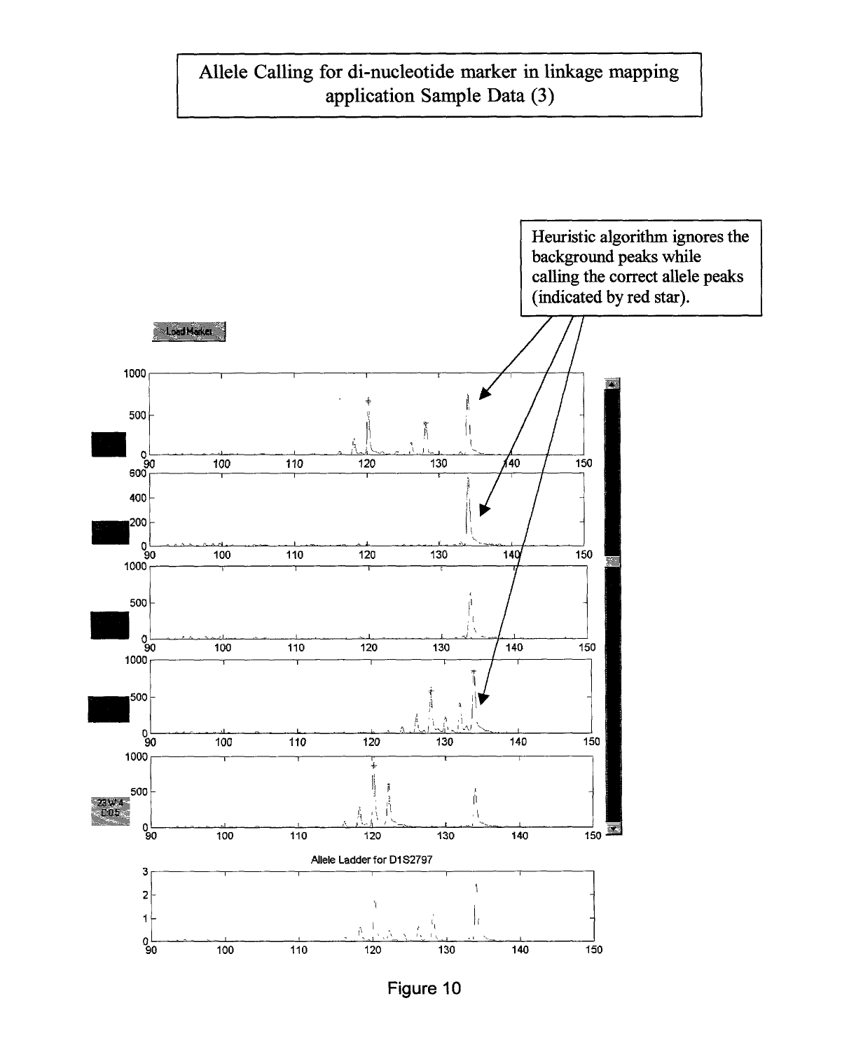

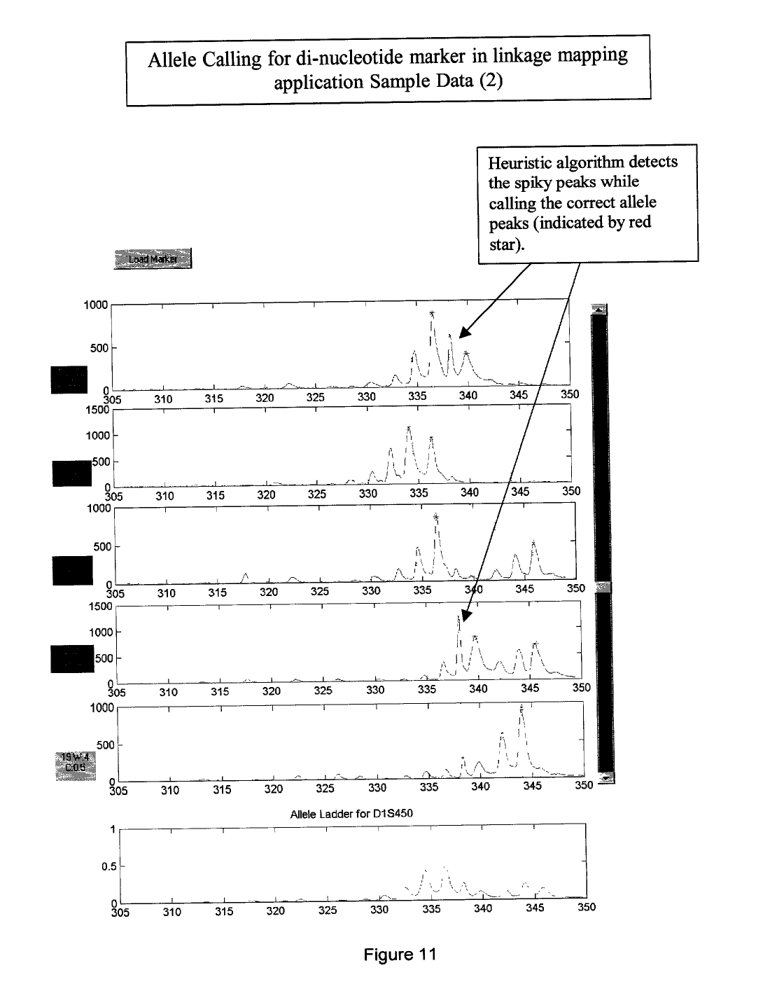

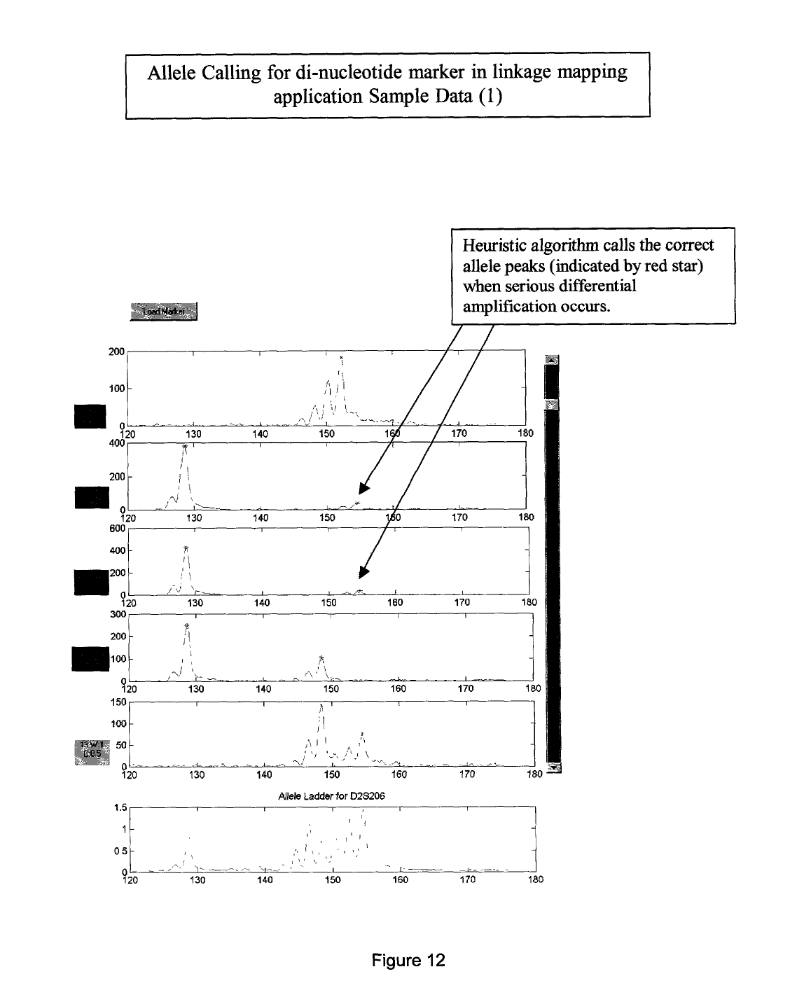

FIGS. 10 through 12 depict data that can be evaluated with the heuristic algorithm according to certain embodiments.

In certain embodiments, the heuristic caller assumes that there are a maximum of two alleles for a given marker. In certain embodiments, there is no such assumption for a maximum number of alleles for a given marker.

V. Committee Machine Processing

The following examples A and B illustrate the Committee approach according to certain embodiments of the invention.

### Example A

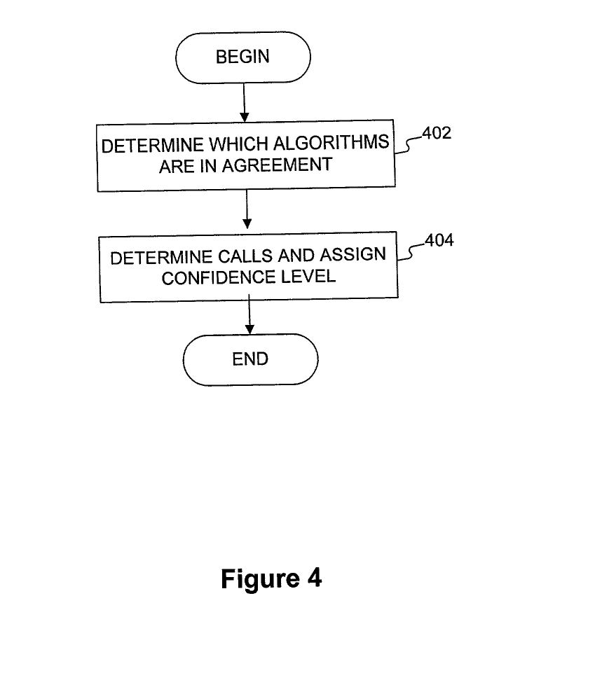

FIG. 4 depicts the steps performed by committee machine 110 according to certain embodiments when determining the final allele calls to be reported to the user and their associated confidence values. Committee machine 110 arbitrates the calls by using a set of rules. An exemplary rule table (Table 2) is depicted below. First committee machine 110 determines which callers are in agreement (step 402).

Next, committee machine 110 determines the correct calls to transmit and assigns a confidence level for these calls (step 404). According to certain embodiments, the confidence level is determined by considering the various cases in Table 2 over a large sample set that is representative of typical data. For example, if all three algorithms are in agreement (case 1), the committee machine assumes that the call is 99.9% correct and thus assigns a confidence value of 0.999. If there is no call for Envelope caller, and the same call for the Optimizer and Heuristic callers, committee machine 110 defines the confidence value as 0.970. If there is no call for the Heuristic algorithm, and the same call for the Envelope method and the Optimizer, committee machine 110 passes those calls to the user and assigns a confidence value of 0.621. If only the Optimizer produces a call, committee machine 110 assigns a confidence value of 0.692 correct. And finally, any cases that do not fit into the above scenarios are assigned the calls given by the Heuristic algorithm and are assigned a confidence value of 0.771. The above listed determination of agreement is exemplary. One skilled in the art will appreciate that other determinations of confidences are available. For example, additional algorithms may be used to produce more accurate confidence levels according to certain embodiments.

TABLE 2

| Results from callers | Confidence |
|---|---|
| Same call by all three algorithms | 0.999 |
| Same call by Optimizer and Heuristic Algorithms | 0.970 |
| No call made by Envelope Caller | |
| Same call by Envelope Caller and Optimizer | 0.621 |
| No call made by Heuristic | |
| Only the Optimizer calls | 0.692 |
| Any cases that do not fit into above categories are | 0.771 |
| called by the Heuristic Algorithm | |

Confidence levels can also be assigned by a person who is familiar with use of the particular algorithms used in a committee approach and the results obtained. Drawing on their experience with the particular algorithms, such a person can assign confidence levels for each of the possible combined results that can be obtained by the various algorithms.

### Example B

1. Allele Calling Algorithms

In this embodiment, three different allele calling algorithms are implemented. Each possesses a distinctly differently philosophy. The callers are

envelope: Only classifies heterozygous data below a level of complexity. It may do so with an extremely high level of accuracy and uses a visual approach based on detection of the characteristic envelop of a relatively noise-free, strong heterozygous signal with good separation between the alleles. If the data looks problematic, envelope refuses to make a call.

optimizer: Uses a maximum likelihood approach involving the formulation of hypotheses based on parameterization of an allele signal using allele location, amount of stutter and +A artifact. The hypothesis that best explains the signal's energy content is declared the winner and the allele calls are those used in forming the winning hypothesis.

heuristic: A rule-based system of allele calling. Initially, all peaks are designated alleles and expert rules are used to remove false candidates until only the true alleles remain. A section devoted to each method follows.

a. Heuristic Caller

Certain programs implement Genotyper allele calling algorithm (ABI PRISM™ Genotyper® 2.0 User's Manual. PE Applied Biosystems, 1996. 850 Lincoln Centre Drive, Foster City, Calif. 94404) and reuse this algorithm for trinucleotide and tetrinucleotide markers during allele calling processes. The steps involved in the process are outlined below.

1. Locate peaks. Find and identify all peaks in the marker size range.

2. Label peaks. Declare all peaks alleles.

3. Global cutoff. Find the maximum peak. Any peak lower than a threshold is removed from the called alleles list. This threshold is determined as cutoffValue * the peak's maximum height where cutoffvalue is a user defined parameter.

4. ⁺A removal. For any two neighboring peaks, if the distance between the peaks is within a certain number (user parameter +A distance) and the ratio between the upstream peak's height and the downstream peak's height exceeds the user parameter ⁺A ratio, the downstream peak is deleted from the called alleles.

5. Stutter removal. For any neighboring two peaks, if the distance between the peaks is within the user parameter stutter distance and the ratio between the downstream peak's height and the upstream peak's height is exceeds the user parameter stutter ratio, the upstream peak is deleted from the called alleles list.

b 6. Declare alleles. Any remaining peaks are declared to be alleles.

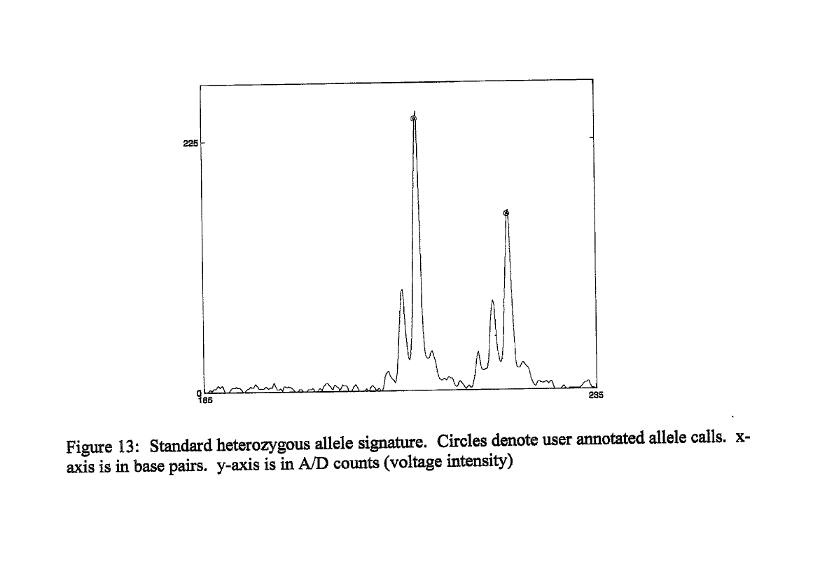

FIG. 13 illustrates a typical standard heterozygous allele signature. (Circles denote user annotated allele calls. x-axis is in base pairs. y-axis is in A/D counts (voltage intensity)) The algorithms behave relatively well for clean dinucleotide marker data and very well for tetrinucleotide marker data. For trinucleotide markers, however, there is a lack of data and it is not known for sure how this algorithm will behave. In all likelihood however, it will probably perform very well.

Certain embodiments of this algorithm include five parameters: cutoffValue, ⁺A distance, ⁺A ratio, stutter distance and stutter ratio. The program provides default values for these parameters and allows the user to adjust these values in the User Interface.

In reviewing large amounts of dinucleotide marker data, it became evident that several situations existed where the Genotyper algorithm was not optimal. These situations constituted the vast majority Genotyper errors. These cases are

1. Differential amplification. One allele is much higher than another allele. The global cutoff rule removes the lower allele.

2. lbp allele. Two alleles exist being separated by only one base pair.

3. Bleedthrough (pullup) peak. Peaks exists due to strong neighboring color peaks and multicomponenting inaccuracy. This may be less than optimal for HID applications.

4. Background peak. One single background peak exists due to poor gel slabs.

5. Spiky stutter peak. Abnormally high and narrow stutter peaks.

The heuristic algorithm addresses these potential sources of error.

The heuristic algorithm includes additional rules. According to certain embodiments, these rules use the combination of feature variables (peak height, peak width, peak begin position, peak end position, peak begin height, peak end height, peak height ratios among peaks, base pair intervals among peaks) to figure out which peaks should be called alleles. In certain embodiments, the algorithm proceeds as follows.

1. Noise Checker. The noise level in the signal is checked. If the signal is too noisy, the process is interrupted.

2. Split Peak Checker. The neighboring peaks are checked for splitting. If splitting exists, only the higher peak is preserved.

3. Background Peak Checker. The peaks are checked to see whether they are single background peaks.

4. Small/Shoulder Peak Checker. Insignificant peaks and/or shoulder peaks are removed.

5. Spike Peak Checker. Spikey stutter peaks are removed

6. ⁺A Checker. The ⁺A peaks are removed.

7. Stutter Checker. The stutter peaks are removed.

8. Special Peak Checker. The peaks are checked to see whether there is differential amplification.

9. Preferential amplification, or if one basepair alleles exist.

These additional rules perform very well and reduce the number of errors substantially.

b. Optimizer Caller

This calling strategy in this embodiment may rest on the assumption that a reasonable model for an allele's signature can be used to build an approximation to the original signal. This approximation is then subtracted from the original signal. The estimate that yields the lowest residual error gives the location of the allele(s).

In examining allele signatures, PCR stutter and ⁺A distortion modify what would ideally be isolated peaks. These, coupled with noise, make locating alleles peak problematic. FIG. 13 illustrates their effect on the signal. Here, PCR stutter appears as a series of diminishing peaks to the left of the main signals at 212 bp and 223 bp and the ⁺A distortion appears as a small peak on the right of the main lobes.

Assuming that the PCR stutter peaks decrease at a constant percentage and assigning a value to the ⁺A distortion, a simple model of the allele signature is parameterized using the following three pieces of information:

allele location;

allele height;

percentage stutter.

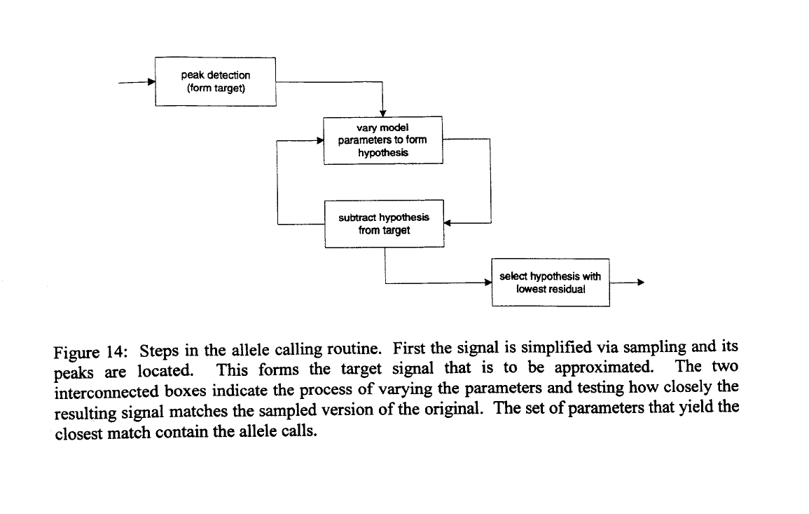

Thus, a search space is created where one considers all combinations of these parameters for a series of candidate allele peaks and obtains their resulting images. These images may then be subtracted from the original signal and the set of parameters with the lowest residual is considered the winner. In this way, the allele locations are identified. The process according to these embodiments is flowcharted in FIG. 14.

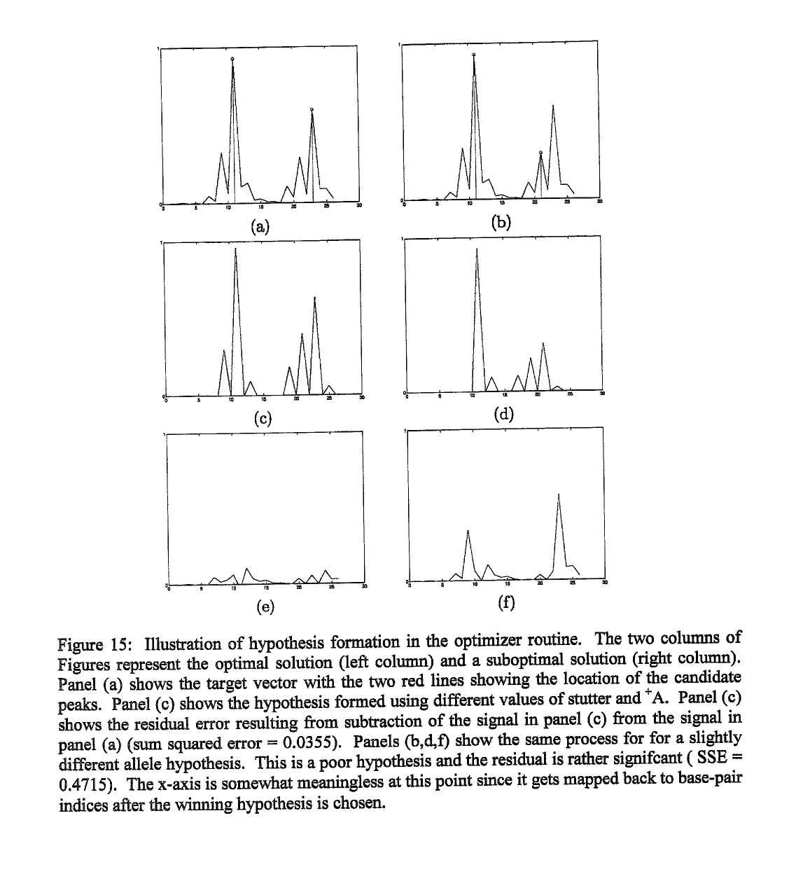

In these embodiments, preprocessing simply involves sampling the original signal to reduce its dimensionality. This can be performed by calculating the most important features of the signal; the peaks and valleys. By representing the signal in such a compact form, the search space is reduced significantly. The peaks form the set of candidate allele peaks that will be considered as possibilities for the allele calls. After the preprocessing, the next two boxes show the varying the parameters and the calculation of the residual. This process is iterated, and in the final box, a winning set of allele peaks (it could be a set of one peak) is declared. Actual output of the algorithm is contained in FIG. 15.

The frames presented here demonstrate two cases; the first (frames (a, c, e)) being the optimal solution and the second (column formed by frames (b, d, f)), shows a solution that while close, does not explain the signal very well and leaves a high residual error. In both cases, the top frame show the signal that is being approximated. The candidate alleles are given by the position of the red lines. The middle frames show the hypothesized signal given different stutter parameters. And finally, the bottom frames show the resulting residual. The column of images on the right clearly demonstrates a better hypothesis and thus is declared the winning hypothesis. Allele calls are given by the locations of proposed peaks (red lines).

c. Envelope Caller

The Envelope caller is developed on the principle that while other callers may generally make a call no matter what, the envelope caller will only call alleles if it determines that there is a high probably that it will be correct. It may be extremely accurate when it makes a call. This boosts the confidence in the calls and removes an entire class of data from requiring further consideration. Its basis is in considering the envelope of the signal and should two large masses of energy be detected (two large humps in the signal), the data is determined to be heterozygous. Allele calling is then simply performed by finding the maximum peak in each hump. While some simple heuristic rules could be added to slightly increase the accuracy. Specifically, these could cover the handful of cases where mistakes are made. However, in certain embodiments, these additional heuristics typically are omitted and instead, the combination of all callers is used to increase confidence to the close to one hundred percent mark in this subset of the data. In certain embodiments, the calling strategies should be fundamentally different in order that they each display strengths for particular data and thus the addition of heuristic rules to this caller may cause it to lose its identity in such embodiments.

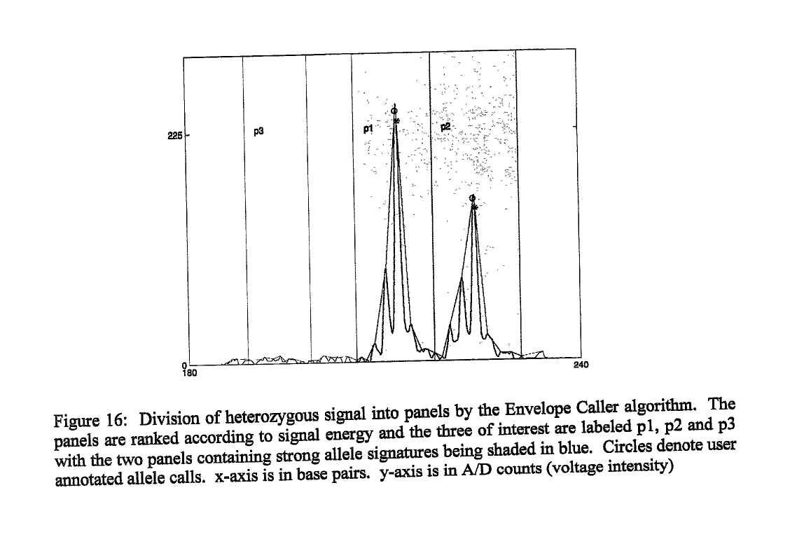

The process is illustrated according to certain embodiments in FIG. 16. The signal has been broken into 6 panels and the energy calculated. Panels marked p1 and p2 are shaded to indicate that they contain the most energy. Energy is denoted E and is the sum of the signal squared. The panel marked p3 contains the third largest energy content. In certain embodiments, the algorithm proceeds to make a call if the following two criteria are met

$$\begin{matrix}\frac{{E}_{p2}}{{E}_{p1}} > 0.2 & ( 1 ) \\ \frac{{E}_{p3}}{{E}_{p2}} < 0.07 & ( 2 )\end{matrix}$$

The call is made by finding the maximums in each of panels 1 and 2. The values of 0.2 and 0.07 in equations 1 and 2 were determined via trial and error and appear to give a good separation between easily classified data and more ambiguous cases.

2. Combination Strategy

In certain instances, the individual algorithms may not be optimal when employed alone. In the committee of experts approach, the degree of confidence for a call is based on the statistical probability of an answer being correct given the degree on consensus between the different callers. This is a particularly apt approach when one considers that one of the callers according to this embodiment only makes a call if it considers it justified. In this embodiment, data falls into one of the following five categories.

Same call for envelope, optimizer, heuristic: The three algorithms are in agreement. This leads to a highly reliable result.

Envelope fails to call, optimizer and the heuristic agree: The signal has been deemed to be more difficult to classify and the process is left to the two more sophisticated approaches. The result is shown to be quite reliable however it is somewhat less confident than above particularly for “bad” data.

Heuristic failed to call, others agree: Sometimes, the heuristic algorithm will not call. This is particularly true in the case of noisy data. In such cases, when agreement between Envelope and the optimizer occurs, that result is presented and the confidence value is defined as the probability that such situations are correct.

Only the optimizer calls: This covers the situation where the data is so problematic that neither Envelope nor the heuristic algorithm calls.

Any data notpreviously called: Should data not be called in the above cases, it is passed to the heuristic routine for calling. Experiment has shown that this algorithm typically surpasses the optimizer in terms of its accuracy when working in isolation.

Results

Results on two series of data from different labs is given in Table 3.

TABLE 3

| | Lab 1 | | | Lab 2 | | | Lab 3 | | |
|---|---|---|---|---|---|---|---|---|---|
| strategy | examples | correct | conf | examples | correct | conf | examples | correct | conf |
| same R1, R2, R3 | 44.2 | 99.99 | 0.999 | 24.6 | 99.9 | 0.999 | 26.1 | 99.99 | 0.999 |
| no R1, same R2 R3 | 51.3 | 99.40 | 0.994 | 58.8 | 97.2 | 0.972 | 70.0 | 99.69 | 0.997 |
| no R3, same R1 R2 | 0.00 | 0.00 | na | 0.5 | 62.1 | 0.621 | 0.2 | 89.29 | 0.893 |
| only R2 calls | 0.04 | 66.7 | 0.667 | 0.8 | 69.1 | 0.691 | 0.3 | 80.00 | 0.800 |
| straglers R2 | 4.5 | 21.2 | 0.212 | 15.2 | 30.9 | 0.309 | 3.5 | 39.67 | 0.400 |
| straglers R3 | 4.5 | 73.6 | 0.736 | 15.2 | 77.1 | 0.771 | 3.5 | 38.45 | 0.385 |

Table 3: Results illustrating confidence values that are created by considering agreement between the calling algorithms. R1—envelope, R2—optimizer, R3—heuristic. All columns are percent-ages except for conf. examples—percentage of examples in full data set that belong to the category strategy, the column correct gives the percentage of examples in that category that are correct. conf is the confidence value, it is percentage correct for a given category. Total number of traces examined: Lab 1—10724, Lab2—8000, Lab 3—14192.

All numbers (except the confidence values) are percentages. The column labeled examples is the percentage of the data set that has fallen into that category. The next two columns recount the percentage of the data from column one that has been correctly and incorrectly classified. The percentage correct has been passed to the column conf to be used as a confidence value. One other casual observation is that lab two possesses data that is distinctly more difficult to process. This can be seen by the number of examples that have fallen through to the final level of processing. This data is marked straglers. Straglers include situations that do not fit into the any of the four categories listed above them in Table 3. For instance, situations in which different algorithms provide an inconsistent allele calls would be considered straglers. Since the data in FIG. 3 shows, in this data, that the call made by algorithm R3 is correct more than the call made by algorithm R2 in such situations, the system may use the results of R3 as a default algorithm when there is inconsistency in the allele call results of algorithms R2 and R3.

The final two rows are for the same chunk of data. They show that the default caller should be the heuristic as it has a higher percentage of correct calls.

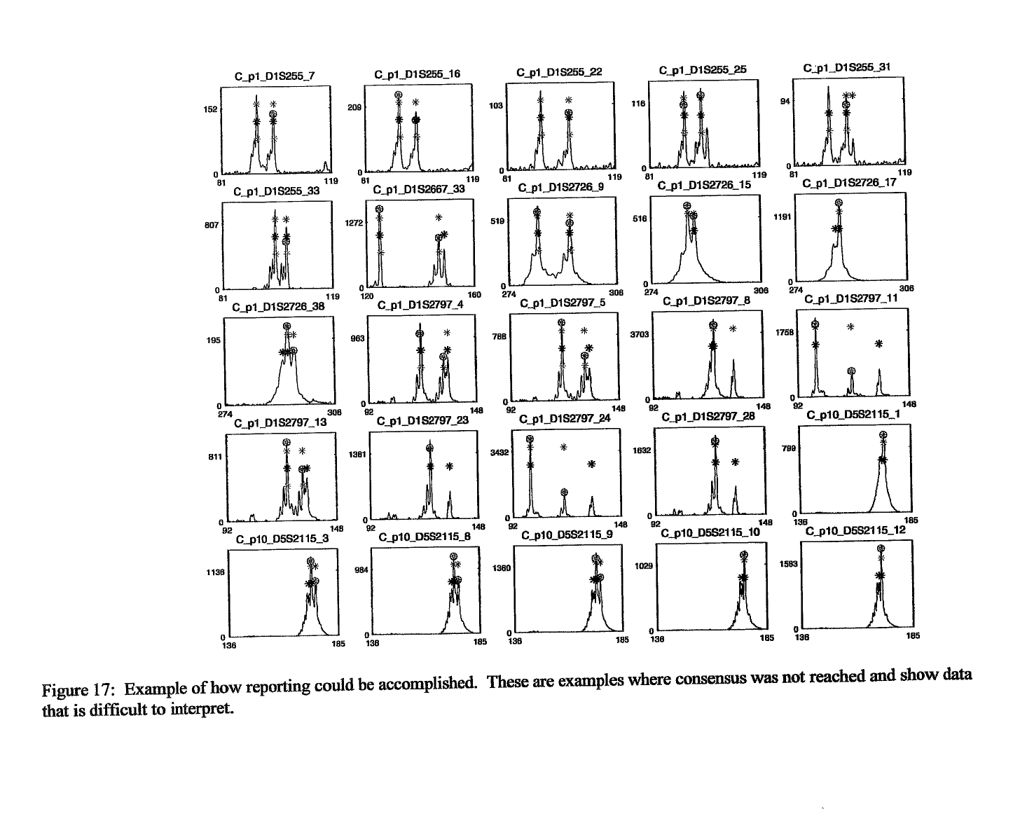

Another interesting opportunity is to pass these results on to the customer as a report—particularly in the case of examples that have fallen into the “difficult to classify” category where no consensus exists. This could be in the form of FIG. 17 and would provide a good visual aid for data checking. FIG. 17 illustrates 25 markers, and though in some cases it appears that consensus was reached, it is not marked as such because the threshold to determine the “sameness” of calls was set too low. In most of the cases however, it can be seen why the data is problematic. The red circles give the user annotations while the three levels of asterisks give the calls for envelope, the optimizer, and the heuristic from bottom to top.

Conclusion

The multi-caller approach is significant in that it provides hard numbers for the confidence in the calls. As well, by partitioning the data into different categories based on how easily the data is classified, it does well in providing a method for checking results.

It is very important to keep in mind that the three methods should not be considered as competing. Rather, as they are based on entirely different philosophies, they serve to confirm each other. The heuristic caller has a vast amount of domain knowledge behind it. The optimizer employs a more formal detection and estimation framework whereby the hypotheses are formed about the allele locations and similar to maximum likelihood, the hypothesis that best explains the signal's energy is chosen as the most likely explanation. Envelope employs a very simple visual inspection to identify easily classified data. These three algorithms each have their strengths and when working in concert form a very robust system and the high degree of trust it is able to place in a call is by virtue of the fact that high confidence requires consensus from a variety of perspectives.

VI. Architecture

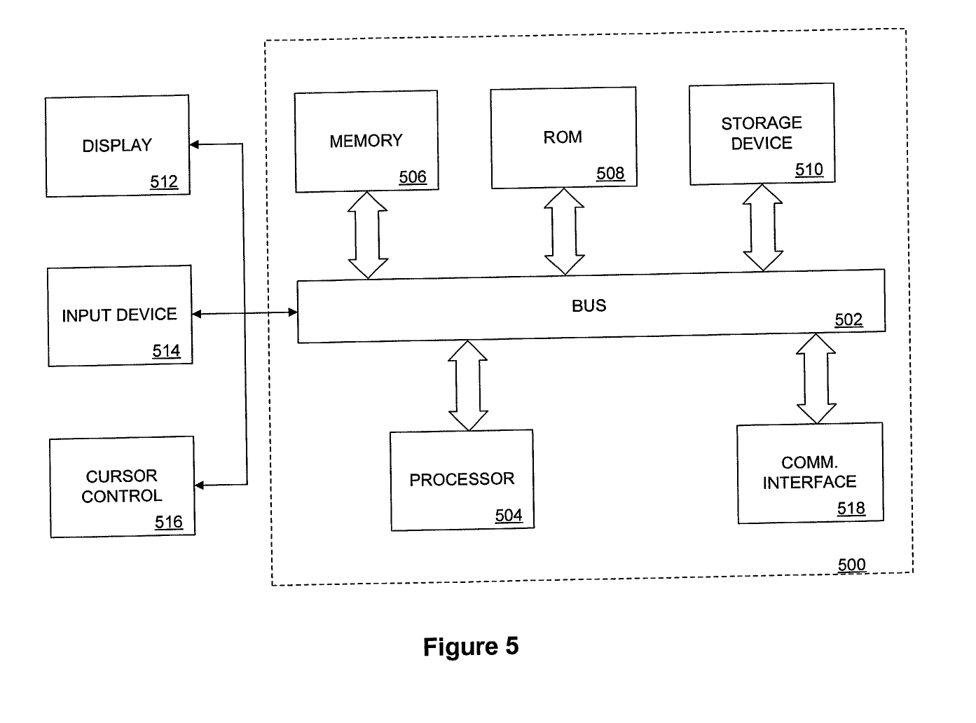

FIG. 5 is a block diagram that illustrates a computer system 500, according to certain embodiments, upon which embodiments of the invention may be implemented. Computer system 500 includes a bus 502 or other communication mechanism for communicating information, and a processor 504 coupled with bus 502 for processing information. Computer system 500 also includes a memory 506, which can be a random access memory (RAM) or other dynamic storage device, coupled to bus 502 for determining allele calls, and instructions to be executed by processor 504. Memory 506 also may be used for storing temporary variables or other intermediate information during execution of instructions to be executed by processor 504. Computer system 500 further includes a read only memory (ROM) 508 or other static storage device coupled to bus 502 for storing static information and instructions for processor 504. A storage device 510, such as a magnetic disk or optical disk, is provided and coupled to bus 502 for storing information and instructions.

Computer system 500 may be coupled via bus 502 to a display 512, such as a cathode ray tube (CRT) or liquid crystal display (LCD), for displaying information to a computer user. An input device 514, including alphanumeric and other keys, is coupled to bus 502 for communicating information and command selections to processor 504. Another type of user input device is cursor control 516, such as a mouse, a trackball or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

A computer system 500 provides allele calls and provides a level of confidence for the various calls. Consistent with certain implementations of the invention, a level of confidence for an allele call is provided by computer system 500 in response to processor 504 executing one or more sequences of one or more instructions contained in memory 506. Such instructions may be read into memory 506 from another computer-readable medium, such as storage device 510. Execution of the sequences of instructions contained in memory 506 causes processor 504 to perform the process states described herein. Alternatively hard-wired circuitry may be used in place of or in combination with software instructions to implement the invention. Thus implementations of the invention are not limited to any specific combination of hardware circuitry and software.

The term “computer-readable medium” as used herein refers to any media that participates in providing instructions to processor 504 for execution. Such a medium may take many forms, including but not limited to, non-volatile media, volatile media, and transmission media. Non-volatile media includes, for example, optical or magnetic disks, such as storage device 510. Volatile media includes dynamic memory, such as memory 506. Transmission media includes coaxial cables, copper wire, and fiber optics, including the wires that comprise bus 502. Transmission media can also take the form of acoustic or light waves, such as those generated during radio-wave and infra-red data communications.

Common forms of computer-readable media include, for example, a floppy disk, a flexible disk, hard disk, magnetic tape, or any other magnetic medium, a CD-ROM, any other optical medium, punch cards, papertape, any other physical medium with patterns of holes, a RAM, PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 504 for execution. For example, the instructions may initially be carried on magnetic disk of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector coupled to bus 502 can receive the data carried in the infra-red signal and place the data on bus 502. Bus 502 carries the data to memory 506, from which processor 504 retrieves and executes the instructions. The instructions received by memory 506 may optionally be stored on storage device 510 either before or after execution by processor 504.

As explained, systems consistent with certain embodiments of the present invention provide a committee machine that receives calls as input from at least two different allele calling algorithms. By receiving these calls, the committee machine is able to determine a level of confidence in a variety of conditions.

The foregoing description of certain embodiments of the committee allele calling approach is not exhaustive and does not in any way limit the claimed invention. For example, although the foregoing was primarily described with reference to particular allele calling algorithms, the concepts of the invention could also be applied to any other type of allele calling algorithms, such as TrueAllele from Cybergenetics or the Genetic Profiler program from Molecular Dynamics. When different algorithms are used, one can assign confidence values for the possible combination of results as discussed above, e.g., by analyzing various cases over a large sample set that is representative of the data or by having a skilled person familiar with the algorithms assigning such confidence values based on experience. Additionally, the described implementation includes software but the present invention may be implemented as a combination of hardware and software or in hardware alone. The invention may be implemented with both object-oriented and non-object-oriented programming systems.

BIN ASSIGNING

In certain embodiments, the system uses a bin assigning algorithm. In certain embodiments, it is desirable to match particular called allele data points from a sample to particular known allele sizes in a population. In certain embodiments, such known allele sizes are already provided. A bin typically is composed of a center point of the known allele size and a given plus and minus value from the center point. Thus, for example, a bin according to certain embodiments will include a center point of a known allele size and include 0.5 points on either side of the center point. Thus, if a data point from a sample falls within that bin, it is assigned to that bin and is given a value of the center point of that bin. If an allele does not locate within any bins, it is assigned as an unknown allele. Bins can be any appropriate size.

In certain embodiments, it is possible to develop a statistical approach to assign the bins. Assuming the population frequency of each bin is known and the alleles within each bin are normally distributed, a simple Bayesian approach can be used to assign the bins.

AUTO BINNING

In certain embodiments, the system includes an auto binning algorithm. In such embodiments, one can determine allele bins for a given population. In certain embodiments, such an algorithm may be used to establish alleles size bins when no allele sizes are yet known for a population. In certain embodiments, such an algorithm may be used to add more allele bins to already known allele size information in a population. For example, one may use an auto binning component to determine additional allele size bins for a population when alleles have been called that do not fall within known allele bins for that population.

In certain embodiments, the auto binning algorithm collects all alleles that have been called for a population and automatically assigns each allele a given bin center based on the allele's data point. In certain embodiments, the algorithm also calculates a cost for each allele which is based on the distance between the data point of the allele and its assigned center bin value. Thus, the further a data point falls from the assigned bin center, the higher the cost. In certain embodiments, the auto binning algorithm then calculates a total cost for all of the alleles. If the total cost is below a certain threshold level, then the chosen bin centers are finalized. If the total cost is above that threshold level, the auto binning algorithm reassigns bin centers to each allele and calculates the total cost in an iterative process until the total cost is below the threshold level. In certain embodiments, after the final bin centers are determined, a given value on either side of the bin centers is added to obtain the final bin. In certain embodiments, 0.5 is added to each side of the bin center to obtain the final bin width.

In certain embodiments, the auto binning algorithm uses a classic k-means clustering algorithm for auto binning. In certain embodiments, the algorithm collects all the alleles from the input samples, removes the alleles whose quality values are less than certain number (0.1) (see quality value discussion below) and feeds them into a iteration process to find the bins as shown in Table 4 below.

TABLE 4

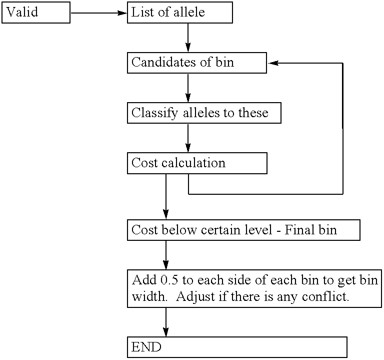

A quality value is then generated based on some large data set research for these newly generated bins (see Quality Value discussion below).

ALGORITHM SUBGROUPS

In certain embodiments, the system includes a preprocessing algorithm, which comprises at least one of an offscale detection algorithm, a multicomponenting algorithm, and a baselining algorithm.

In certain embodiments, the system includes a data conversion algorithm, which comprises at least one of a peak detecting algorithm, a size standard matching algorithm, a ladder shift algorithm, and a size calling algorithm.

In certain emboments, the system includes a allele call reporting algorithm, comprising at least one of an allele calling algorithm, an auto binning algorithm, and a bin assigning algorithm.

ALLELE CALL REPORT

In certain embodiments, the allele call report is the reported allele call that may be provided after an allele calling algorithm has been applied. In certain embodiments, the allele call report may be provided after an allele calling algorithm and one or more subsequent algorithms have been applied. For example, in certain embodiments, the allele call report may be provided after an allele calling algorithm and a subsequent bin assigning algorithm have been applied. In certain embodiments, the predicted accuracy of an allele call report may be generated in view of certain quality values as discussed below. In certain embodiments, the predicted accuracy is a predicted that the allele call report is correct.

QUALITY VALUES AND WARNING FLAGS

In certain embodiments, the system uses one or more quality values (QVs) and/or a warning flags. In certain embodiments, quality values may be used to predict the accuracy of the called alleles in the data. In certain embodiments, quality values and/or warning flags may be used to predict the accuracy of the allele call report. In certain embodiments, the predicted accuracy is a prediction of whether or not the allele call report is correct. In certain embodiments, if the quality value falls below a given threshold, one will be prompted to check the data again. In certain embodiments, if the quality value falls below a further threshold, one will be prompted to not consider the data at all. In certain embodiments, the predicted accuracy provides a value for predicting whether an allele call report is correct.

Quality values may be used for any or all of the algorithms within a system. Exemplary quality values are discussed below.

MULTICOMPONENTING QV

In certain embodiments, the system uses a multicomponenting QV to determine the quality value of the multicomponenting result. In certain embodiments, one may employ methods such as those discussed in U.S. Pat. No. 6,015,667, to Sharaf, Issued Jan. 18, 2000, which incorporated by reference herein.

BASELINING QV

In certain embodiments, the system uses a baselining QV to determine the quality value of the baselining result. For instance, in certain embodiments, if a maximum likelihood model fit method is used for baselining, with the baseline as one component of the model and the fragment peaks as other components, the residual signal is an indication of the error of the baseline.

SIZE STANDARD MATCHING QV

In certain embodiments, the system uses a size standard matching QV to determine the quality value of the size standard matching result. In certain embodiments, the size standard matching QV is determined using two processes. The first process is that it calculates the scan number base pair ratio or scaling factor (which is the inverse of the ratio) from the matching result. If this ratio is larger than 0.25 (in other words, the scaling factor is less than four scans per one base pair), the matching result is not correct and the quality value is 0.0. In certain embodiments, the second process is based on chi-square test. From the size standard definition, it calculates the theoretical (expected) distances (in base pair) among all these fragments. From the matched peaks, it calculates the observed distances (in base pair) among these mapped fragrnents. A chi-square test is performed to see whether these two sets of distances is similar enough. The P-value of this test is reported as the quality value of the matching.

An example of this process follows. In the process, one is determining the Chi square value for the hypothesis that the observed peak distribution does not differ from expected. This may be calculated in the following way according to certain embodiments.

After sizing, peaks have two values: Size (the size of the peak) and scan number (the time when the peak was scanned). Assume that the following data was obtained:

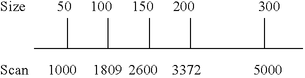

One may use the two outer peaks, to determine the scaling factor.

In this case

$$\frac{1000 - 5000}{300 - 50} = 16 scans per base .$$

For each pair of peaks one can calculate the observed basepair distance between them. For the data above, e.g., between the 50 and 100 bp peaks, one expects to see a 50 basepair distance, but the observed basepair distance is 809/16=50.5625 basepair.

By calculating this for each pair of peaks, one can calculate the Chi square value for the hypothesis that the observed peak distribution does not differ from expected. The P value obtained is then used as the size quality value. The other flags typically have no effect on this.

ALLELE CALLING QV

In certain embodiments, the system uses an allele calling QV to determine the quality value of an allele calling algorithm. In certain embodiments, the more than one allele calling algorithm is employed, and an allele calling QV is based on the results obtained from the more than one allele calling algorithm. In certain embodiments, an allele calling QV that is based on the results for more than one allele calling algorithm is called a consensus value or consensus quality value.

In certain embodiments, an allele calling QV is generated for each allele calling algorithm. One skilled in the art will be able to determine processes for generating quality values for various allele calling algorithms. In certain embodiments, one may generate an overall allele calling QV for the combination of allele calling algorithms by averaging the quality values for each allele calling algorithm that makes an allele call. In certain embodiments, one may generate an overall allele calling QV for the combination of allele calling algorithms by choosing the minimum individual quality value of the allele calling algorithms that make an allele call. In certain embodiments, one may generate an overall allele calling QV for the combination of allele calling algorithms by choosing the maximum individual quality value of the allele calling algorithms that make an allele call. In certain embodiments, if only one of the allele calling algorithms makes an allele call, the quality value of that allele calling algorithm may be used as the overall quality value.

In certain embodiments in which more than one allele calling algorithm is applied, an allele calling quality value may be generated in view of the percent of correct calls that fit into various categories of consensus between the different allele calling algorithms. For example, one may process a large number of samples with known alleles with the different allele calling algorithms. One then determines the percent of correct allele calls when there is a consensus of all of the allele calling algorithms, and when there are various different levels of consensus, e.g., when certain algorithms make a call and others do not. One can then generate an allele calling QV based on those percentages.

In certain embodiments, one may have one lab perform all of the work and the percent of correct allele calls for each category is used for the QV. Thus, 99% of the allele calls are correct when all of the allele calling algorithms call an allele, , a QV of 0.99 is used when all algorithms make a call in subsequent work. If 75% of the allele calls are correct when algorithms A and B agree, and algorithm C does not make a call, a QV of 0.75 is used when such a result is obtained in subsequent work.

In certain embodiments, one can determine an allele calling QV by having more than one lab generate such data. In certain embodiments, one may then average the QV's for each category of results that are obtained from the different labs. In certain embodiments, one may then use the minimum QV for each category of results that are obtained from the different labs. In certain embodiments, one may then use the maximum QV for each category of results that are obtained from the different labs.

In certain embodiments, one may use for the allele calling QV the confidence values that are discussed above when the envelope caller, the optimizer caller, and the heuristic caller are employed together. For example, in certain embodiments, one may use the confidence values set forth in Tables 2 or 3 above.

HEURISTIC QV

In certain embodiments, the heuristic allele calling algorithm uses some heuristic rules to generate a reasonable (based on a large test data), but subjective quality value qvH for its allele calling process. Certain embodiments employ the following rules:

1. Quality value starts with value 1.0;

2. For the Noise Checker, the quality value is multiplied by (1.0—noiseLevel);

3. For the Special Peak Checker, the quality value is multiplied by a series of 0.5 if the algorithm decides the signal contains peculiar stutter patterns and peculiar multiple peak patterns.

4. The quality value is further decreased if the called allele peaks violate the user settable peak height ratio, peak absolute height, and broad peak thresholds.

AUTO BINNING QV

In certain embodiments, the system uses an auto binning QV to determine the quality value of the auto binning component. In certain embodiments, the auto binning QV is determined during the auto binning process. In certain embodiments, after finding all the bin centers, the auto binning component iterates through all the alleles involved and bin centers to calculate the residue (mean square error). This residue is adjusted by the marker repeat. This adjusted residue AR is used as a determinant for binning quality value. In certain embodiments, from a large data set research, the following rules are found. If AR is less than 0.30, the binning is good, and no manual inspection is needed and the quality value is assigned to be 1.0. If AR is between 0.30 and 0.40, the binning is likely good, and some bins need to be checked and quality value is assigned to be 0.50. If AR is larger than 0.40, binning is unacceptable, and there could be some mistakes in the allele sizes, and all bins need to be checked and quality values is assigned to be 0.0. Also, in certain embodiments, if the user sets the bins without employing an auto binning component, the quality value is set at 1.0.

BIN ASSIGNING QV

In certain embodiments, the system uses a bin assigning QV to determine the quality value of the assignment of sample alleles to set bins. In certain embodiments, the bin assigning QV is determined by the distance given alleles are located from the bin centers. In certain embodiments, the bin assigning QV is set at 1 if the allele falls within a bin, and is set at 0.1 if the allele does not fall within a bin.

ALLELE CALLING WARNING FLAGS

In certain embodiments, the system reports to the user multiple warning flags. The warning flags alert the user that there could be potential problems with the accuracy of the data. Certain embodiments employ the following warning flags:

Offscale—The flag is set when there is an offscale peak within the calling range. (The calling range is calculated after size calling has been performed.)

Spiky Peak—The flag is set when there is spiky peak present in the marker signal. In certain embodiments, the flag is set if the narrowest peak in a cluster has a width 50% less than the neighboring peak.

One Basepair Peak—The flag is set when there is one basepair allele present in the marker signal. For example, the flag is set in certain embodiments when there are two called alleles that are separated by only one base pair.

Peak Height Ratio—The flag is set when there are two alleles present and the ratio between lower allele height and higher allele height is below certain level. In certain embodiments, this level is set to 0.5.

Peak Absolute Height—The flag is set when the alleles are lower than the specified values. In certain embodiments, these values are set to 200 if homozgyous and 100 if heterozygous.

Binning problem—The flag is set when the called alleles are not assigned into any of the user-defined bins.

Bleedthrough—The flag is set when the marker signal contains bleed through peaks (pull up peaks). In certain embodiments, bleed through is detected when there is a peak in a different color within 1 scan and that peak is less than 20% of the larger one.

Broad Peak—The flag is set when the called alleles' peak width is wider than a certain value. In certain embodiments, this value is set to 1.5 base pair. In certain embodiments, one measures the peak width at half of the peak's height.

Background Peak—The flag is set when the marker signal contains single (lone) peaks. In certain embodiments, a background peak is one that does not fit into a cluster. In certain embodiments, a background peak is determined to exist when there is a small peak beside a large peak, which does not fit the pattern of a microsatellite. Such background peaks may occur due to some error in the slab gel electrophoresis.

Split Peak—The flag is set in certain embodiments if the following data is obtained:

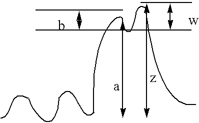

a/b>10 and z/w>10 and distance between two peaks is <0.25 base pair. or

a/b>8 and z/w>40 and distance between two peaks is <0.25

The higher peak is used as the real allele.

Number of Allele Error—The flag is set when the number of alleles exceed the maximum number possible for the species or no alleles are found.

The following Table 5 shows certain embodiments of the invention that employ various warning flags:

TABLE 5
Summary of which Flags are used (● = used; and a blank = not used)

| | Spiky | Back- | One | | | PA | Bleed | Allele | Broad | Split | |
|---|---|---|---|---|---|---|---|---|---|---|---|
| | peak | ground | Base | Binning | PHR | H | through | error | Peak | Peak | OGQ |
| Linkage Di | ● | ● | ● | ● | ● | ● | | ● | ● | ● | ● |
| Nucleotides | | | | | | | | | | | |
| Linkage Tris, | | | | ● | ● | ● | | ● | ● | | ● |
| Tetras | | | | | | | | | | | |
| HID | | | | ● | ● | ● | ● | ●* | ● | | ● |
| Tris, Tetras | | | | | | | | | | | |

Offscale is also used for all three (Linkage Dinucleotide, Linkage Tris and Tetras, and HID Tris and Tetras) according to certain embodiments.
*Not used for allelic ladder samples

ALLELE CALL REPORT QV

In certain embodiments, the system uses an allele call report QV (also called an overall quality value) to determine the quality value of the allele call report. (As discussed above, the allele call report may be provided after an allele calling algorithm and a bin assigning algorithm have been applied.)

In certain embodiments, one may generate an allele call report quality value based on an integrated quality value from a series of individual algorithm component quality values.

qvAllele=qvSizeMatch×qvAllelePeakPick×qvBinAssign×qvBin

qvSizeMatch comes from the size matching algorithmn.

QvAllelePeakPick comes from the allele peak picking algorithm. It may be a consensus value if the system uses more than one allele peak picking algorithm.

QvBinAssign comes from the bin assigning algorithm.

QvBin comes from the setting of the bins for a population. In certain embodiments, this value is generated by the auto binning algorithm. But if the bin is specified by the user, the qvbin is 1.0.

In certain embodiments, one may generate an allele call report quality value based on any or all of the following quality values. In certain embodiments, one may generate an allele call report quality value by multiplying two or more of the individual values or flags that are to be used as follows.

Size Matching QV

Allele Peak Picking QV (In certain embodiments, it may be the consensus value, which is a percentage based on internal calibrations. In certain embodiments involving ladders, the Marker Quality Value may be used rather than the consensus value.)

Bin Assigning QV

Auto Binning QV

If any of the following flags are set, a multiple of 0.5 is used for each instance: Background Peak, Offscale, Peak Height Ratio, Peak Absolute Height. In the case of Peak Height Ratio, if the lower height allele is to the left, one uses a multiple of 0.25 instead of 0.5.

If there is an Number of Allele Error flag, the quality value is set at 0.

If the user manually edits any of data that would have been impacted by a quality value, the value is set at 1 for the factor that is edited.

In certain embodiments, one can average all of the allele quality values to give a genotype quality value when more than two alleles are present. In such embodiments, each allele has its own quality value and one averages all of those quality values to obtain a genotype quality value.

In certain embodiments, one may generate an allele call report QV based on a mean value of several individual generated allele call report QV's for the same marker.

HUMAN IDENTIFICATION

In certain embodiments, the system is used for human identification. In certain such embodiments, there are certain given markers that include different known alleles at each marker for a given population. For each marker, the known alleles are provided to the user as a ladder to which the generated data can be compared. The ladder is a sample that includes differently sized nucleotides, each corresponding to a particular allele for a given marker.

The user is also apprised of bins that have bin centers that each correspond to the expected size of each of the differently sized nucleotides for each allele in the ladder. From run to run and instrument to instrument, when one employs the ladders in a process, there may exist some shifts for these ladder locations. In other words, the data generated when one uses the ladders in an experiment may include ladder peak sizes that do not correspond exactly with the expected bin centers and may include more peaks than expected bin centers. Thus, in certain embodiments, one may use a ladder shift algorithm to adjust the bin locations to account for these ladder shifts and/or additional peaks to provide bins that may provide more accurate results for determining the size of alleles in an experimental sample than unadjusted bin locations.

In certain embodiments, to figure out the ladder shifts, the system finds the locations of the ladders (by searching bin definitions, which are the expected bin centers for the alleles of a ladder that are reported to the user) and uses a dynamic programming algorithm to match the bin locations to the peaks of the ladder signal. In certain embodiments, one can use the size standard matching algorithm discussed above to account for the shift in the ladders and/or the extra peaks by matching bin definitions (the reported expected bin centers) with the actual peaks obtained with the ladder files. In certain embodiments, the matching algorithm employs a minimum peak height of 100 to 150 rfu since the ladders typically are very strong signals.). After matching, the shifts are calculated for each ladder bin definition/peak pair.

Each ladder is then provided revised bins for assigning peaks obtained from a sample. For example, after the system calls alleles in a sample, the alleles are assigned to bins that have been adjusted using the shifts.

According to certain embodiments, the process proceeds using the flow chart in Table 5:

TABLE 5

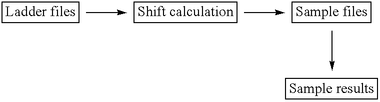

In certain embodiments, the size standard matching component discussed above is used for ladder shifts as follows. In these embodiments, alleles within a given ladder are assigned to bins. In certain embodiments, the user is also alerted to virtual bins. Virtual bins are bins in which an allele may occur, but that possible allele is not provided in the ladder. In certain embodiments, the virtual bins may need to be shifted when there is a shift determined for the actual alleles in the ladder. In the following description, the shifts are detected for the ladder of each marker independently from other ladders for other markers.

In certain embodiments, the size standard matching algorithm discussed above in the Size Standard Matching section is used to evaluate the data generated with the ladders by matching the peaks expected in the ladder to the peaks actually observed (in certain embodiments, one uses peaks>100 rfu).

If fewer peaks are observed than the number expected for a given ladder for a particular marker, then one should not use the ladder for that marker in the analysis (note that such results are determined independently for each ladder for each marker).

If more peaks are observed than the number expected for a given ladder for a particular marker, then the size standard matching component will attempt to fit the observed pattern to the expected pattern.

If the matching is successful, in certain embodiments, it will generate a marker quality value (also called a ladder shift quality value). In certain embodiments, the marker quality value is generated using the same technique that is discussed above in the Size Standard Matching QV section. (This marker quality value may be used instead of the allele calling quality value in the overall genotyping quality.)

Note that an extra peak will not necessarily generate a lower quality value.

The algorithm is now aware of which ladder peak represents which bin. It takes the allelic ladder peak size calculated above and subtracts from it the value of the expected bin center. This gives a bin shift for that bin in that allelic ladder file. Any virtual bins are given the same shift as the closest ladder bin to its left. Thus, if a ladder file has a shifted an allele bin center +0.2 from the expected bin center, a virtual bin to the right of such a ladder bin will also have its center shifted +0.2.

In certain embodiments, this shift is calculated for each marker, and bin shifts from each ladder file are calculated and stored. In certain embodiments, a given ladder is run more than once in the process. In such embodiments, one can average any bin shifts by averaging the individual bins across the ladders. For example, assume that a bin in marker X has a shift of +1, +2 and +0 in three separate sample ladders for marker X. The average shift would be +1). (Note there is no check on whether these bin shifts cause overlapping bins.) Also, note that averaging is across all ladder files used in a single run. In certain embodiments, an individual run is all files in the same folder

Using Bin Shifts

After a peak is determined to be an allele in a test sample, the peak size is then compared to the shifted bins to determine which bin in which it should be placed. When the test allele falls within one bin, one can then conclude that such an allele corresponds to the particular allele of the ladder corresponding to that bin. If the allele can be assigned with more than one bin or no bins, the allele is labeled as an off-ladder allele.

SYSTEM COMPONENTS ACCORDING TO CERTAIN EMBODIMENTS

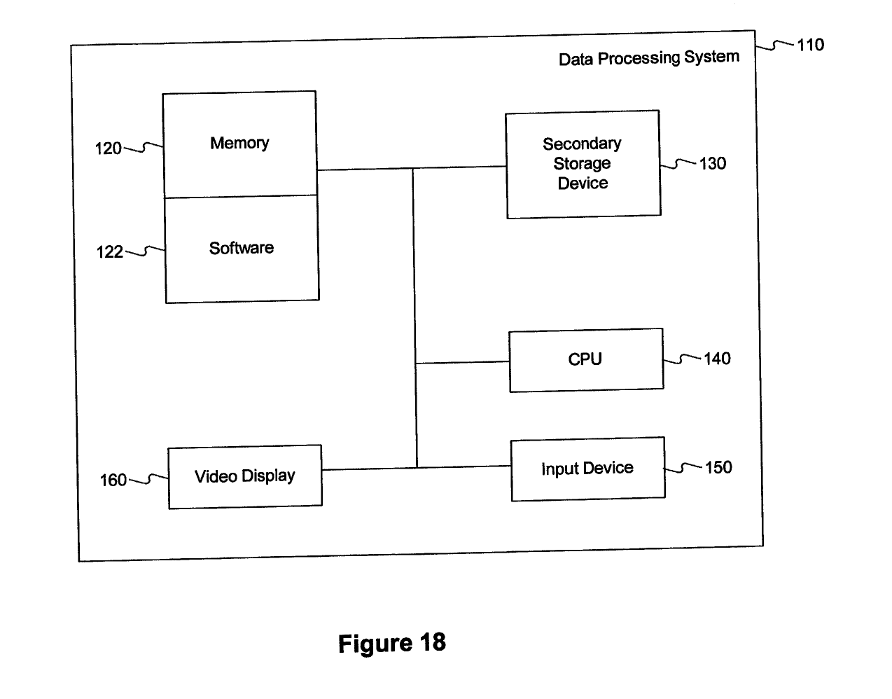

FIG. 18 depicts a more detailed diagram of data processing system 100 for use with certain embodiments. System 100 contains a memory 120, a secondary storage device 130, a central processing unit (CPU) 140, an input device 150, and a video display 160. Memory 120 includes software 122 containing algorithms for matching in-lane size standards with its definition and algorithms for linkage mapping markers and human identification markers.

Although aspects of the present invention are described as being stored in memory, one skilled in the art will appreciate that these aspects may be stored on or read from other computer-readable media, such as secondary storage devices, like hard disks, floppy disks, and CD-ROM; a carrier wave received from a network like the Internet; or other forms of ROM or RAM. Additionally, although specific components and programs of system 100 have been described, one skilled in the art will appreciate that it may contain additional or different components or programs.

1. A computer-implemented method for making allele calls, comprising:
receiving data representing nucleic acid information;
applying at least two different allele calling algorithms to the data to provide a result for each algorithm; and
depending on agreement between the results of each algorithm, identifying an allele call within the data and assigning a confidence level for each call.

2. The computer-implemented method of claim 1, wherein the allele calling algorithms applied in the step of applying at least two different allele calling algorithms to the data to provide a result for each algorithm are selected from an envelope detection caller algorithm, an optimizer caller algorithm, and a heuristic caller algorithm.

3. A computer-implemented method for making allele calls, comprising:
receiving a signal representing nucleic acid information;
determining whether the signal is below a predefined complexity; and
making an allele call for the signal based on the determination.

4. A computer-implemented method for making allele calls, comprising:
receiving signal representing nucleic acid information;
applying a set of filters to the signal to eliminate peaks that do not represent alleles, wherein the set of filters include at least one of the following: a split peak checker; a background peak checker; a shoulder peak checker; a spike peak checker; a special peak checker; and a one basepair checker; and
determining that remaining peaks in the data are alleles after applying the set of filters to the signal.

5. The method of claim 4, wherein the applying step includes the substeps of:
creating a list of peaks in the signal;
determining characteristics associated with each peak; and
removing peaks from the list based on the determined characteristics.

6. The method of any of claims 1, 3, or 4, wherein the nucleic acid information is nucleic acid length.

7. A computer-implemented method for interpreting nucleotide or amino acid information, comprising:
receiving data representing nucleotide or amino acid information;
applying at least two different algorithms to the data to provide a result for each algorithm; and
depending on agreement between the results of each algorithm, identifying at least one correct result within the data and assigning a confidence level to the at least one correct result.

8. The computer-implemented method of claim 7, wherein the algorithms applied in the step of applying at least two different algorithms to the data to provide a result for each algorithm are selected from an envelope detection caller algorithm, an optimizer caller algorithm, and a heuristic caller algorithm.

9. A computer-implemented method for making allele calls associated with data representing nucleic acid information, comprising:
applying each one of a plurality of allele calling algorithms to data representing nucleic acid information to determine whether there are any allele calls represented in the data, wherein each allele calling algorithm applies a different strategy in determining whether there is an allele call represented in the data;
if results from all of the applied allele calling algorithms are consistent, assigning a high level of confidence for any allele calls identified in the data during application of the allele calling algorithms;
if results from all of the applied allele calling algorithms are not consistent, assigning different levels of confidence for any allele calls identified in the data during application of the allele calling algorithms depending upon which combination of the applied allele calling algorithms share consistent results; and
outputting a report including information associated with the results and any assignment of confidence levels for any allele calls identified in the data.

10. The computer-implemented method of claim 9, wherein the allele calling algorithms applied in the applying each one of a plurality of allele calling algorithms to data representing nucleic acid information to determine whether there are any allele calls represented in the data, wherein each allele calling algorithm applies a different strategy in determining whether there is an allele call represented in the data, are selected from an envelope detection caller algorithm, an optimizer caller algorithm, and a heuristic caller algorithm.

11. A system for making allele calls, comprising:
a processor configured to execute program instructions; and
a memory containing program instructions for execution by the processor to
receive data representing nucleic acid information,
apply at least two different allele calling algorithms to the data to provide a result for each algorithm, and
depending on agreement between the results of each algorithm, identify an allele call within the data and assigning a confidence level for each call.

12. The computer-implemented method of claim 1 1, wherein the allele calling algorithms applied are selected from an envelope detection caller algorithm, an optimizer caller algorithm, and a heuristic caller algorithm.

13. The system of claim 11, wherein the nucleic acid information comprises nucleic acid length.

14. A system for making allele calls, comprising:
a processor configured to execute program instructions; and
a memory containing program instructions for execution by the processor to
receive a signal representing nucleic acid information,
determine whether the signal is below a predefined complexity, and
make an allele call for the signal based on the determination.

15. The system of claim 14, wherein the nucleic acid information comprises nucleic acid length.

16. A system for making allele calls, comprising:
a processor configured to execute program instructions; and
a memory containing program instructions for execution by the processor to
receive signal representing nucleic acid information,
apply a set of filters to the signal to eliminate peaks that do not represent alleles, wherein the set of filters include at least one of the following: a split peak checker; a background peak checker; a shoulder peak checker; a spike peak checker; a special peak checker; and a one basepair checker, and
determine that remaining peaks in the data are alleles after applying the set of filters to the signal.

17. The system of claim 16, wherein when the processor executing program instructions applies the set of filters to the signal to eliminate peaks that do not represent alleles, the processor creates a list of peaks in the signal, determines characteristics associated with each peak, and removes peaks from the list based on the determined characteristics.

18. The system of claim 16, wherein the nucleic acid information comprises nucleic acid length.

19. A system for interpreting nucleotide or amino acid information, comprising:
a processor to execute program instructions; and
a memory that stores program instructions for execution by the processor to
receive data representing nucleotide or amino acid information,
apply at least two different algorithms to the data to provide a result for each algorithm, and
depending on agreement between the results of each algorithm, identify at least one correct result within the data and assigning a confidence level to the at least one correct result.

20. The system of claim 19, wherein the algorithms applied are selected from an envelope detection caller algorithm, an optimizer caller algorithm, and a heuristic caller algorithm.

21. A system for making allele calls associated with data representing nucleic acid information, comprising:
a processor to execute program instructions; and
a memory that stores program instructions for execution by the processor to
apply each one of a plurality of allele calling algorithms to data representing nucleic acid information to determine whether there are any allele calls represented in the data, wherein each allele calling algorithm applies a different strategy in determining whether there is an allele call represented in the data,
if results from all of the applied allele calling algorithms are consistent, assign a high level of confidence for any allele calls identified in the data during application of the allele calling algorithms,
if results from all of the applied allele calling algorithms are not consistent, assign different levels of confidence for any allele calls identified in the data during application of the allele calling algorithms depending upon which combination of the applied allele calling algorithms share consistent results, and
output a report including information associated with the results and any assignment of confidence levels for any allele calls identified in the data.

22. The system of claim 21, wherein the allele calling algorithms applied are selected from an envelope detection caller algorithm, an optimizer caller algorithm, and a heuristic caller algorithm.

23. A computer readable medium containing instructions for controlling a computer system to perform a method for making correct allele calls, the method comprising:
receiving data representing nucleic acid information;
applying at least two different allele calling algorithms to the data to provide a result for each algorithm; and
depending on agreement between the results of each algorithm, identifying an allele call within the data and assigning a confidence level for each call.

24. The computer readable medium of claim 23, wherein the allele calling algorithms applied in the applying of at least two different allele calling algorithms to the data to provide a result for each algorithm are selected from an envelope detection caller algorithm, an optimizer caller algorithm, and a heuristic caller algorithm.

25. A computer readable medium containing instructions for controlling a computer system to perform a method for making allele calls, the method comprising:
receiving a signal representing nucleic acid information;
determining whether the signal is below a predefined complexity; and
making an allele call for the signal based on the determination.

26. A computer readable medium containing instructions for controlling a computer system to perform a method for making allele calls, the method comprising:
receiving signal representing nucleic acid information;
applying a set of filters to the signal to eliminate peaks that do not represent alleles, wherein the set of filters include at least one of the following: a split peak checker; a background peak checker; a shoulder peak checker; a spike peak checker; a special peak checker; and a one basepair checker; and
determining that remaining peaks in the data are alleles after applying the set of filters to the signal.

27. The computer readable medium of claim 26, wherein the applying of the set of filters includes:
creating a list of peaks in the signal;
determining characteristics associated with each peak; and
removing peaks from the list based on the determined characteristics.

28. The method of any of claims 23, 25, or 26, wherein the nucleic acid information is nucleic acid length.

29. A computer readable medium containing instructions for controlling a computer system to perform a method for interpreting nucleotide or amino acid information, the method comprising:
receiving data representing nucleotide or amino acid information;
applying at least two different algorithms to the data to provide a result for each algorithm; and
depending on agreement between the results of each algorithm, identifying at least one correct result within the data and assigning a confidence level to the at least one correct result.

30. The computer readable medium of claim 29, wherein the algorithms applied are selected from an envelope detection caller algorithm, an optimizer caller algorithm, and a heuristic caller algorithm.

31. A computer readable medium containing instructions for controlling a computer system to perform a method for making allele calls associated with data representing nucleic acid information, the method comprising:
applying each one of a plurality of allele calling algorithms to data representing nucleic acid information to determine whether there are any allele calls represented in the data, wherein each allele calling algorithm applies a different strategy in determining whether there is an allele call represented in the data;
if results from all of the applied allele calling algorithms are consistent, assigning a high level of confidence for any allele calls identified in the data during application of the allele calling algorithms;
if results from all of the applied allele calling algorithms are not consistent, assigning different levels of confidence for any allele calls identified in the data during application of the allele calling algorithms depending upon which combination of the applied allele calling algorithms share consistent results; and
outputting a report including information associated with the results and any assignment of confidence levels for any allele calls identified in the data.

32. The computer readable medium of claim 31, wherein the allele calling algorithms applied in the applying of each one of a plurality of allele calling algorithms to data representing nucleic acid information to determine whether there are any allele calls represented in the data, wherein each allele calling algorithm applies a different strategy in determining whether there is an allele call represented in the data, are selected from an envelope detection caller algorithm, an optimizer caller algorithm, and a heuristic caller algorithm.

33. A system for making allele calls, comprising:
means for receiving data representing nucleic acid information;
means for applying at least two different allele calling algorithms to the data to provide a result for each algorithm; and
means for depending on agreement between the results of each algorithm, identifying an allele call within the data and assigning a confidence level for each call.

34. A computer-implemented method for obtaining an allele call report, comprising:
receiving data representing nucleic acid information;
applying at least two different algorithms to the data to provide an allele call report;
generating a first algorithm quality value based on one of the at least two different algorithms;
generating a second algorithm quality value based on another of the at least two different algorithms;
generating an allele call report quality value based on at least the first and second algorithm quality values; and
predicting the accuracy of allele call report in view of the generated allele call report quality value.

35. The computer-implemented method of claim 34, wherein the applying of the at least two different algorithms comprises applying at least two of the following algorithms a) through c):
a) a preprocessing algorithm, comprising at least one of an offscale detection algorithm, a multicomponenting algorithm, and a baselining algorithm;
b) a data conversion algorithm, comprising at least one of a peak detecting algorithm, a size standard matching algorithm, and a size calling algorithm; and
c) an allele call reporting algorithm, comprising at least one of an allele calling algorithm, an auto binning algorithm, and a bin assigning algorithm.

36. The computer-implemented method of claim 35, wherein the generating ofthe first and second quality values comprises generating a quality value for the data conversion algorithm and generating a quality value for the allele call reporting algorithm.

37. The computer-implemented method of claim 35, wherein the applying of the at least two different algorithms comprises applying:
a data conversion algorithm, comprising at least one of a peak detecting algorithm, a size standard matching algorithm, and a size calling algorithm; and
an allele call reporting algorithm, comprising at least one of an allele calling algorithm, an auto binning algorithm, and a bin assigning algorithm.

38. The computer-implemented method of claim 37, wherein the generating of the first and second algorithm quality values comprises generating a quality value for the data conversion algorithm and generating a quality value for the allele call reporting algorithm.

39. The computer-implemented method of claim 35, wherein the applying of the at least two different algorithms comprises applying:
a data conversion algorithm, comprising a peak detecting algorithm, a size standard matching algorithm, and a size calling algorithm; and
an allele call reporting algorithm, comprising an allele calling algorithm.

40. The computer-implemented method of claim 39, wherein the generating of the first and second algorithm quality values comprises generating a quality value for the data conversion algorithm by a process comprising generating a quality value for the size standard matching algorithm, and generating a quality value for the allele call reporting algorithm by a process comprising generating a quality value for the allele calling algorithm.

41. The computer-implemented method of claim 35, wherein the applying of the at least two different algorithms comprises applying:
a data conversion algorithm, comprising a peak detecting algorithm, a size standard matching algorithm, and a size calling algorithm; and
an allele call reporting algorithm, comprising an allele calling algorithm and a bin assigning algorithm.

42. The computer-implemented method of claim 41, wherein the generating of the first and second algorithm quality values comprises generating a quality value for the data conversion algorithm by a process comprising generating a quality value for the size standard matching algorithm, and generating a quality value for the allele call reporting algorithm by a process comprising generating a quality value for the allele calling algorithm.

43. The computer-implemented method of claim 42, wherein the process for generating a quality value for the allele call reporting algorithm further comprises generating a quality value for the bin assigning algorithm, and generating the quality value for the allele call reporting algorithm based on the quality value for the allele calling algorithm and the quality value for the bin assigning algorithm.

44. The computer-implemented method of claim 35, wherein the applying of the at least two different algorithms comprises applying:
a data conversion algorithm, comprising a peak detecting algorithm, a size standard matching algorithm, and a size calling algorithm; and
an allele call reporting algorithm, comprising an allele calling algorithm, an auto binning algorithm, and a bin assigning algorithm.

45. The computer-implemented method of claim 44, wherein the generating of the first and second algorithm quality values comprises generating a quality value for the data conversion algorithm by a process comprising generating a quality value for the size standard matching algorithm, and generating a quality value for the allele call reporting algorithm by a process comprising generating a quality value for the allele calling algorithm.

46. The computer-implemented method of claim 45, wherein the process for generating a quality value for the allele call reporting algorithm further comprises generating a quality value for the bin assigning algorithm, and generating the quality value for the allele call reporting algorithm based on the quality value for the allele calling algorithm and the quality value for the bin assigning algorithm.

47. The computer-implemented method of claim 46, wherein the process for generating a quality value for the allele call reporting algorithm further comprises generating a quality value for the auto binning algorithm, and generating the quality value for the allele call reporting algorithm based on the quality value for the allele calling algorithm, the quality value for the bin assigning algorithm, and the quality value of the auto binning algorithm.

48. The computer-implemented method of claim 34, wherein the applying of the at least two different algorithms comprises applying:
a preprocessing algorithm, comprising at least one of an offscale detection algorithm, a multicomponenting algorithm, and a baselining algorithm;
a data conversion algorithm, comprising at least one of a peak detecting algorithm, a size standard matching algorithm, and a size calling algorithm; and
an allele call reporting algorithm, comprising at least one of an allele calling algorithm, an auto binning algorithm, and a bin assigning algorithm.

49. The computer-implemented method of claim 48, wherein the generating of the first and second algorithm quality values comprises generating a quality value for the data conversion algorithm and generating a quality value for the allele call reporting algorithm.

50. The computer-implemented method of claim 49, further comprising generating a third algorithm quality value, which comprises generating a quality value for the preprocessing algorithm, and generating an allele call report quality value based on at least the first, second, and third algorithm quality values.

51. The computer-implemented method of claim 48, wherein the applying of the at least two different algorithms comprises applying:
a preprocessing algorithm, comprising at least one of an offscale detection algorithm, a multicomponenting algorithm, and a baselining algorithm;
a data conversion algorithm, comprising a peak detecting algorithm, a size standard matching algorithm, and a size calling algorithm; and
an allele call reporting algorithm, comprising an allele calling algorithm and a bin assigning algorithm.

52. The computer-implemented method of claim 51, wherein the generating of the first and second algorithm quality values comprises generating a quality value for the data conversion algorithm by a process comprising generating a quality value for the size standard matching algorithm, and generating a quality value for the allele call reporting algorithm by a process comprising generating a quality value for the allele calling algorithm.

53. The computer-implemented method of claim 52, wherein the process for generating a quality value for the allele call reporting algorithm further comprises generating a quality value for the bin assigning algorithm, and generating the quality value for the allele call reporting algorithm based on the quality value for the allele calling algorithm and the quality value for the bin assigning algorithm.

54. The computer-implemented method of claim 35, wherein the applying of the at least two different algorithms comprises applying:
a data conversion algorithm, comprising a peak detecting algorithm, a size standard matching algorithm, and a size calling algorithm; and
an allele call reporting algorithm, comprising applying at least two different allele calling algorithms to provide a result for each algorithm.

55. The computer-implemented method of claim 54, wherein the generating of the first and second algorithm quality values comprises generating a quality value for the data conversion algorithm by a process comprising generating a quality value for the size standard matching algorithm, and generating a quality value for the allele call reporting algorithm by a process comprising generating a quality value for the allele calling algorithm based on the results of each of the at least two different allele calling algorithms.

56. The computer-implemented method of claim 34, wherein the applying of the at least two different algorithms comprises applying:
a data conversion algorithm, comprising a peak detecting algorithm, a ladder shift algorithm, and a size calling algorithm; and
an allele call reporting algorithm, comprising an allele calling algorithm and a bin assigning algorithm.

57. The computer-implemented method of claim 56, wherein the generating of the first and second algorithm quality values comprises generating a quality value for the data conversion algorithm by a process comprising generating a quality value for the ladder shift algorithm, and generating a quality value for the allele call reporting algorithm by a process comprising generating a quality value for the bin assigning algorithm.

58. The computer-implemented method of claim 34, wherein the applying of the at least two different algorithms comprises applying at least two of the following algorithms:
an offscale detection algorithm;
a multicomponenting algorithm;
a peak detecting algorithm;
a baselining algorithm;
a size standard matching algorithm;
a size calling algorithm;
an allele calling algorithm;
an auto binning algorithm; and
a bin assigning algorithm.

59. The computer-implemented method of claim 58, wherein the applying of the at least two different algorithms comprises applying a baselining algorithm, a size standard matching algorithm, a size calling algorithm, an allele calling algorithm, and a bin assigning algorithm.

60. The computer-implemented method of claim 59, wherein the generating of the first and second algorithm quality values comprises generating a quality value for a size standard matching algorithm and an allele calling algorithm.

61. The computer-implemented method of claim 60, further comprising generating a third algorithm quality value, which comprises generating a quality value for the bin assigning algorithm, and generating an allele call report quality value based on at least the first, second, and third algorithm quality values.

62. A system for making allele calls, comprising:
a processor configured to execute program instructions; and
a memory containing program instructions for execution by the processor to
receive data representing nucleic acid information,
apply at least two different algorithms to the data to provide an allele call report;
generate a first algorithm quality value based on one of the at least two different algorithms;
generate a second algorithm quality value based on another of the at least two different algorithms;
generate an allele call report quality value based on at least the first and second algorithm quality values; and
predict the accuracy of allele call report in view of the generated allele call report quality value.

63. A computer readable medium containing instructions for controlling a computer system to perform a method of making allele calls, the method comprising:
receiving data representing nucleic acid information;
applying at least two different algorithms to the data to provide an allele call report;
generating a first algorithm quality value based on one of the at least two different algorithms;
generating a second algorithm quality value based on another of the at least two different algorithms;
generating an allele call report quality value based on at least the first and second algorithm quality values; and
predicting the accuracy of allele call report in view of the generated allele call report quality value.
